SEQUENCE LISTING (9 sequences)

SEQ ID NO: 1 - DNA, 41 bp, Artificial Sequence
  Description of Artificial Sequence Synthetic primer
gggggatccaaaaaatggatggtactatggttagcaagggc

SEQ ID NO: 2 - DNA, 35 bp, Artificial Sequence
  Description of Artificial Sequence Synthetic primer
cccactagtttaagatctcttgtacagctcgtcca

SEQ ID NO: 3 - DNA, 30 bp, Artificial Sequence
  Description of Artificial Sequence Synthetic primer
aaaaggatccatggtattcgttaaaggtca

SEQ ID NO: 4 - DNA, 36 bp, Artificial Sequence
  Description of Artificial Sequence Synthetic primer
tttttactagtttagtttttgctttcagttggattg

SEQ ID NO: 5 - DNA, 40 bp, Artificial Sequence
  Description of Artificial Sequence Synthetic primer
aaaggtaccgaagacttcctgatggtgatgatggttatga

SEQ ID NO: 6 - DNA, 34 bp, Artificial Sequence
  Description of Artificial Sequence Synthetic primer
tttgtcgacattcaaatgattcaccagtgcagtt

SEQ ID NO: 7 - DNA, 34 bp, Artificial Sequence
  Description of Artificial Sequence Synthetic primer
aaaggatccagacattcccaaagaatatgactgt

SEQ ID NO: 8 - DNA, 40 bp, Artificial Sequence
  Description of Artificial Sequence Synthetic primer
tttactagtgaagacaaaaaaaatttcccgctttgaaggg

SEQ ID NO: 9 - protein, 6 aa, Artificial Sequence
  Description of Artificial Sequence Synthetic 6xHis tag
HHHHHH

(19) United States
(12) Patent Application Publication
(10) Pub. No.: US 2020/0197451 A1
(43) Pub. Date: Jun. 25, 2020

(54) LECTIN-BOUND BACTERIA AND USES THEREOF

(71) Applicant: Baylor College of Medicine, Houston, TX (US)

(72) Inventors: Adam Kuspa, Houston, TX (US); Christoper Quang Dung Dinh, Houston, TX (US)

(21) Appl. No.: 16/639,985

(22) PCT Filed: Aug. 17, 2018
(86) PCT No.: PCT/IB2018/056242

§ 371 Date: Feb. 18, 2020

Related U.S. Application Data
(60) Provisional application No. 62/547,391, filed on Aug. 18, 2017.

(51) Int. Cl. A61K 35/745; A61K 35/747; A61K 38/17

(52) U.S. Cl. CPC A61K 35/745; A61K 38/1732; A61K 35/747

(57) ABSTRACT

Embodiments of the disclosure include methods and compositions related to lectin-coated bacteria of any kind and the imparted activity of being resistant to one or more antibacterial agents and or environmental conditions. In at least some cases, lectin-coated bacteria are utilized to improve a microbiome in a subject. The lectin-coated bacteria may also be employed for uptake into cells, including eukaryotic cells such as mammalian cells.

### CROSS-REFERENCE TO RELATED APPLICATIONS

This application claims priority to U.S. Provisional Patent Application Ser. No. 62/547,391, filed Aug. 18, 2017, which is incorporated by reference herein in its entirety.

### STATEMENT REGARDING FEDERALLY SPONSORED RESEARCH OR DEVELOPMENT

This invention was made with government support under 5P01HD039691 awarded by National Institute of Child Health and Human Development and also under PO1 HD39691 from the Dictyostelium Functional Genomics Program Project Grant from the National Institutes of Health. The government has certain rights in the invention.

### TECHNICAL FIELD

Embodiments of the disclosure concern at least the fields of cell biology, microbiology, bacteriology, molecular biology, and medicine.

### BACKGROUND

D. discoideum amoebae live in the soil and feed on bacteria, but as food becomes scarce they aggregate into a mound and form a migrating slug which eventually develops into a fruiting body containing a spore-filled sorus on a cellular stalk (1). It had been thought that development occurred free of bacteria and that the final fruiting body was sterile (2). D. discoideum has an innate immune system comprised of Sentinel cells that are able to rid the slug of interstitial bacteria (3, 4); however, it was recently reported that about one-third of wild isolates maintain symbiotic interactions with the bacteria in their soil environment and carry bacteria stably through cycles of growth and development (5). In a form of microbial farming, carried bacteria can seed a new food supply for germinating spores as they establish a new colony of amoebae (5). Additional interactions between D. discoideum carriers and their bacterial associates have been documented and there is evidence they are controlled by specific signals (6-8). The demonstration herein that amoebal lectins mediate bacterial carriage indicates that D. discoideum actively controls its microbiome, and that amoeba-bacterium interactions are a useful model for microbiome homeostasis.

The disclosure also provides solutions for a long-felt need in the art of providing bacteria effectively to an organism to maintain or improve a healthy microbiome.

### BRIEF SUMMARY

Embodiments of the disclosure include systems, methods, and compositions related to the preparation and use of bacteria having lectins (carbohydrate-binding proteins) bound thereto. The disclosure concerns one or more types of bacteria having one or more types of lectins bound thereto, including in a coat form in at least some cases. In particular embodiments, the lectin-bound bacteria are utilized for methods directed to modifying a microbiome.

Particular aspects to the disclosure include providing effective amounts of lectin-bound bacteria to improve a microbiome, including to increase in a microbiome the levels of one or more types of bacteria compared to normal levels of the one or more types of bacteria or for the purpose of increasing the levels of one or more types of bacteria that are at deficient levels, as examples.

In some embodiments, the disclosure concerns transformation of one or more types of cells, such as eukaryotic cells, including mammalian cells, with one or more types of bacteria having lectins bound thereto.

Embodiments of the disclosure include methods of improving a microbiome in a subject, comprising the step of providing to the subject an effective amount of a composition comprising a plurality of one or more types of isolated bacteria having one or more types of lectins externally associated thereto, said bacteria formulated in a pharmaceutically acceptable carrier. The subject may be of any kind, such as an animal or plant. Examples of animals include livestock animals or farm animals, including mammals. Examples of a mammal include a human, dog, cat, horse, lamb, sheep, pig, goat, rabbit, or cow. The animal may be of any age, including an adult, adolescent, child, or infant, including a newborn. Animals include fish or birds, including those utilized for agriculture. In cases wherein the subject is a plant, the plant may be an agricultural plant or ornamental plant, for example. The subject may be healthy, may have a medical condition, or may be at risk for a medical condition in comparison to the average individual in the population. The medical condition may be a gastrointestinal medical condition or an autoimmune medical condition. Prior to, during, and/or after being provided the composition(s) of the disclosure, the subject may consume one or more prebiotics. In some cases, methods further comprise the step of identifying that the subject is in need of the composition(s) of the disclosure.

Bacteria to be bound to lectin, and/or bacteria bound with lectin may be of any kind, including Gram negative, Gram positive, aerobic, anaerobic, or a mixture thereof. In some cases, the bacteria are nonpathogenic bacteria selected from the group consisting of Bifidobacterium, Clostridium, Fusobacterium, Melissococcus, Propionibacterium, Streptococcus, Enterococcus, Lactococcus, Staphylococcus, Peptostrepococcus, Bacillus, Pediococcus, Micrococcus, Leuconostoc, Weissella, Aerococcus, Oenococcus, Lactobacillus, Bacteriodetes, Prevotella, Bacteroides, Firmicutes., Xylanibacter, Faecalibacterium, Eubacterium, Subdoligranulum, Parabacteriodetes, Ruminococcus, Collinsella, Rosehburia, Akkermansia, Veillonella, Verrucomicrobia, Escherichia, and a mixture thereof. The bacteria may comprise Bifidobacterium and/or Lactobacillus, in particular cases. In specific cases, the bacteria are selected from the group consisting of Bifidobacterium animalis, Escherichia coli, Lactococcus lactis, Lactobacillus reuteri, Tobacillus acidophilus, Acidophilus bifidus, Streptococcus thermophiles, Streptococcus faecium, Clostridium leptum, Clostridium coccoides, Bacillus coagulans, B. lentus, B. licheniformis, B. mesentericus, B. pumilus, B. subtilis, B. natto, Bacteroides amylophilus, Bac. capillosus, Bac. ruminocola, Bac. suis, Bifidobacterium adolescentis, B. animalis, B. breve, B. bifidum, B. infantis, B. lactis, B. longum, B. pseudolongum, B. thermophilum, Candida pintolepesii, Clostridium butyricum, Enterococcus cremoris, E. diacetylactis, E. faecium, E. intermedius, E. lactis, E. muntdii, E. thermophilus, Escherichia coli, Kluyveromyces fragilis, Lactobacillus acidophilus, L. alimentarius, L. amylovorus, L. crispatus, L. brevis, L. case L. curvatus, L. cellobiosus, L. delbrueckii ss. bulgaricus, L. farciminis, L. fermentum, L. gasseri, L. helveticus, L. lactis, L. plantarum, L. johnsonii, L. reuteri, L. rhamnosus, L. sakei, L. salivarius, Leuconostoc mesenteroides, P. cereviseae (damnosus), Pediococcus acidilactici, P. pentosaceus, Propionibacterium freudenreichii, Prop. shermanii, Saccharomyces cereviseae, Staphylococcus carnosus, Staph. xylosus, Streptococcus infantarius, Strep. Salivarius, Strep. thermophilus, Strep. lactis, and a mixture thereof.

Lectins utilized in methods and compositions of the disclosure may be mannose binding lectins, galactose/N-acetylgalactosamine binding lectins, N-acetylglucosamine binding lectins, N-acetylneuraminic acid binding lectins, or Fucose binding lectins, for example. The lectin may be a C type lectin, L type lectin R type lectin, or P type lectin. The lectin may be Galectin, Concanavalin A, collectin, selectin Lentil lectin, Snowdrop lectin, Ricin, Peanut agglutinin, Jacalin, Hairy vetch lectin, Wheat Germ Agglutinin, Elderberry lectin, Maackia amurensis leukoagglutinin, Maackia amurensis hemoagglutinin, Ulex europaeus agglutinin, Aleuria aurantia lectin, BanLec, Achylectin, Anti-B agglutinin, calnexin, calreticulin, or a mixture thereof.

In one embodiment, there is a method of protecting one or more types of bacteria from destruction by one or more antibacterial agents and/or from destruction by an environment, comprising the step of exposing the one or more types of bacteria to an effective amount of one or more types of lectins to produce bacteria having the one or more types of lectins externally associated thereto, whereby upon exposure to the one or more antibacterial agents and/or the environment, said one or more types of bacteria are protected. The antibacterial agent may be a protein or nucleic acid, including from one or more types of pathogenic bacteria. The environment may be a gastrointestinal system of an animal, including an animal in need of improvement of its microbiome whether or not the individual is healthy or has a medical condition, such as a gastrointestinal medical condition.

In some embodiments, there are methods of producing a probiotic composition, comprising the steps of: exposing one or more types of bacteria to one or more types of lectin under conditions sufficient to result in binding of the lectin(s) to the bacteria to produce lectin-bound bacteria; and formulating the lectin-bound bacteria in a pharmaceutically acceptable carrier.

In one embodiment, there is a method of facilitating uptake of one or more types of bacteria into one or more types of cells, comprising the step of binding one or more types of lectins to the bacteria in a sufficient amount for the bacteria to be taken up into the cell. Any lectin encompassed by the disclosure may be derived from one or more plants, amoebae, animals, or a mixture thereof. The bacteria may be Gram-negative or Gram-positive; pathogenic or non-pathogenic; and/or spherical, rod shaped, or spiral. The bacteria may be of the type Coccus, Bacillus, Vibrio, Spirillum, or Spirochete; the bacteria may be E. coli, for example. The bacteria may be genetically engineered, such as comprising at least one genetically engineered mutation. The cells may be of any kind, including eukaryotic; they may be mammalian cells, such as cancer cells, non-cancer cells, immune cells, epithelial cells, primary cell, an established cell line, hybridoma cells, germ cells, somatic cells, stem cells, progenitor cells, pluripotent cells, totipotent cells, or a mixture thereof. Any cell may be a human cell. The cell may be from the ectoderm, mesoderm, or endoderm; any cell may be in culture. The method may be performed occurs ex vivo, in vivo, or in vitro.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawings.

FIGS. 1A-1D. Extracellular killing of bacteria by D. discoideum and secreted antibacterial activity. (1A) Plaques of D. discoideum strain AX4 growing on lawns of K. pneumoniae were stained with fluorescent dyes that distinguish live and dead bacteria as visualized by fluorescence microscopy (upper panel) and DIC microscopy (lower panel). Numbers (1,2) indicate the areas that were sampled to reveal dead (red) and live (green) bacteria (right panels). (1B) Plaques of wild carrier and non-carrier strains stained as in a. (1C) Carrier amoebae (car⁺, circles) and non-carrier amoebae (car⁻; squares, diamonds) were mixed with K. pneumoniae in buffer and the number of viable bacteria were determined over time and normalized to 100 percent at start of the assay. Values are means and standard error of the means (±s.e.m.) for three independent experiments. (1D) Secretomes from QS23 (non-carrier) and QS37 (carrier) were resolved by ion-exchange chromatography (DEAE-Sepharose) with a step-elution of increasing salt (NaCl, dotted line) and the fractions were assayed for antibacterial activity against K. pneumonia. Trace activity is sometimes observed in carrier secretomes that elutes near the peak observed in non-carriers (asterisk). When discoidin I is removed from carrier secretomes, anti-bacterial activity becomes apparent (open circles).

FIGS. 2A-2B. Discoidin I secretion by wild carrier strains. Wild strains were harvested during late-stage growth on lawns of K. pneumoniae bacteria (When most of the bacteria had been consumed), shaken in Sor buffer for 1 hour before being separated into supernatant (S) and pellet (P) fractions and resolved on SDS-PAGE protein gels. (2A) Western blots stained with anti-discoidin I antibodies showing soluble and cell-associated discoidin I in carrier and non-carrier strains. The DscA/C control (left lane) was purified from Ax4 (Supplemental Materials). (2B) A representative time course of discoidin I production from before bacteria are consumed to the start of the growth to development transition, for a non-carrier (QS14) and a carrier (QS68) strain. The 3-hour point is roughly equivalent to the time of harvest in A.

FIGS. 3A-3C. Discoidin I binds and protects bacteria. (3A) Increasing numbers of K. pneumoniae (K.p.) bacteria (1X=2.3×10⁷ bacteria) were incubated with purified DscA (300 μg/ml) in 100 μl buffer for 60 min at room temperature. Bacteria were separated from soluble DscA by centrifugation, and the supernatant (S) and pellet (P) fractions were resolved by SDS poly-acrylamide gel electrophoresis and visualized by coomassie blue staining. (3B) DscA (12.5-62.5 μg) was incubated with 1.3×10⁸ K. pneumoniae, or E. coli, in 100 μl for 60 min. Bacteria were removed by centrifugation and the unbound DscA was determined and used to calculate the amount of DscA bound to the bacteria. (3C) K. pneumoniae bacteria, pretreated with DscA or bovine serum albumin (BSA), were suspended in buffer and challenged with Dab's. Bacterial viability was quantitated by counting live bacteria at various times, using a live-dead staining reagent and fluorescence microscopy, normalized to the zero-time samples. Statistically significant differences (p<0.001, *, **, ***) were observed between DscA-treated bacteria and the BSA-treated bacteria at all time points (one-way pairwise ANOVA using Tukey contrasts for multiple comparisons of means).

FIGS. 4A-4D. Lectin Induced Modified Bacterial Internalization (LIMBI) results in bacterial endosymbiosis, increased persistence of bacteria within in amoebae and genetic transformation. (4A) D. discoideum amoebae (QS4) were mixed with GFP-expressing K. pneumoniae that had been pretreated with buffer (mock) or DscA and allowed to develop into fruiting bodies. Fluorescence microscopy of the resulting spores showing intact bacteria in spores after LIMBI with DscA. (4B) An overlay image of differential interference contrast (DIC) and fluorescence microscopy showing vegetative amoebae and GFP-expressing and DscA-coated E. coli under agar. Their persistence within amoebae was quantified by determining the number of minutes that each bacterium was observed to remain intact. (4C) Box plots of the persistence time of discoidin-coated bacteria within amoebae after LIMBI. The edges of the boxes represent the 75^{th} and 25^{th} percentiles, medians are the thick lines within, the whiskers are the minimum and maximum, and the dots are the outliers. The difference between LIMBI and the no-lectin control was statistically significant (p<0.001, Mann-Whitney-Wilcoxon rank sum test). (4D) LIMBI-mediated gene transfer from E. coli into D. discoideum. Discoidin-coated E. coli harboring a plasmid engineered to express a red fluorescent protein fusion to D. discoideum histone H2b (mCherry-H2b) were mixed with AX4 amoebae followed by 10 days of drug selection for the plasmid. Images by DIC and fluorescence microscopy of nuclear DNA stained with DAPI (DAPI) or mCherry, and an overlay showing nuclear expression of mCherry-H2b.

FIGS. 5A-5C. Bacterial carriage during D. discoideum development. (5A) Migrating slugs of carrier and non-carrier wild isolates of D. discoideum after initiating development with a mixture of amoebae and food bacteria in the center of a non-nutrient agar plate. Note the bacterial colonies that form in the trails behind carrier slugs (left panel, inset box). (5B) An assay for bacterial carriage where the sori are deposited on nutrient agar plates and scored for the presence or absence of bacterial growth after incubation at 37° C., shown in for a carrier (5B, lower left panel) and a non-carrier (5B, lower right panel). (5C) Phase-contrast (left panel) and fluorescent images (right panel) of the contents of the sori of a wild isolate carrier strain after development in the presence of Bacillus subtilis bacteria expressing Green Fluorescent Protein (GFP). Intact bacteria are seen mainly within spores and the arrow indicates two interstitial bacteria.

FIGS. 6A-6F. End-point dilution assay for secreted Dictyostelium antibacterial (Dab) activity and protection by discoidin I. K. pneumoniae bacteria were seeded into wells of 96-well plates containing SM media, to which DEAE-sepharose column eluate fractions from D. discoideum secretomes (FIG. 1D) were added and the plate was incubated at 37° C. overnight. (6A) Tenfold dilutions of peak fractions from the step elution with NaCl were quantified by a twofold dilution series, as shown on the right, and used to quantify yield and purity. Sample elution profiles (12-ml step-elutions with NaCl, 1-ml fractions) are shown for the secretomes of one non-carrier strain (6B) and two carrier secretomes (6C, 6D). Trace activity is sometimes observed in the carrier secretomes that partially inhibits bacterial growth at tenfold dilution (6D, asterisk). (6E) No activity was detected in the carrier strain secretomes unless they were first passed over a Sepharose CL-4B column to remove discoidin I lectins, as described in Results (Table 2). (6F) K. pneumoniae bacteria incubated overnight at 37° C. with dilutions of Dab's produced as described in FIG. 1D (top two rows). An eightfold dilution of Dab's (middle two rows) was used to test the protective effect of DscA/C (bottom two rows) at a level (50 μg/ml) that is saturating for binding to K. pneumonia under these conditions. The protective effect of discoidin I was lost at fourfold dilution (12.5 μg/ml).

FIGS. 7A-7B. Proteins secreted by wild isolates of D. discoideum. (7A) Proteins secreted from wild strains, harvested from bacterial growth plates at the time of the growth to development transition, were resolved by SDS-PAGE and stained with coomassie blue. The carrier strain (car+) bands indicated by a single asterisk contain mainly DscA and DscC (Table 3), and the double asterisks indicate the bands containing mainly CadA (Table 4). (7B) Western blot detection of discoidin I in the supernatants from QS4 (car−) and QS70 (car+). The dilution series of the samples suggests there is >80-times more discoidin I secreted by QS74 compared with QS4.

FIGS. 8A-8C. LIMBI-mediated gene transfer from E. coli into D. discoideum. LIMBI of lectin-coated E. coli harboring a plasmid engineered to express a red fluorescent protein fusion to D. discoideum histone H2b (mCherry-H2b) under control of an actin promoter into AX4 amoebae. (8A) Western blot with mouse monoclonal antibodies directed against mCherry show mCherry-H2b protein expression in DscA LIMBI-transformed amoebae, but no expression in the control population that was maintained without selection. (8B, 8C) LIMBI transformation of amoebae with the mCherry-H2b expression plasmid using plant lectins to coat the E. coli. Overlays of DIC and fluorescence images are shown for LIMBI with ConA (8B) and SBA (8C).

FIGS. 9A-9B. Retention of bacteria within mammalian cells after LIMBI. E. coli bacteria expressing either RFP or GFP were coated with SBA lectin. The lectin-coated bacteria were mixed with uncoated bacteria and added to cultures of RAW264.7 macrophages. The bacteria-cell mixtures were incubated overnight (˜16 hours), the plates were then washed twice with PBS to remove most free-floating bacteria and the cultures imaged on a Nikon inverted microscope (Eclipse Ti) in DIC and fluorescence channels. FIG. 9A demonstrates RAW264.7 cells+E. coli GFP (+SBA) and E. coli RFP (no lectin) and FIG. 9B demonstrates RAW264.7 cells+E. coli GFP (no lectin) and E. coli RFP (+SBA). Note that only the lectin-coated bacteria survive within the macrophages after the incubation.

FIGS. 10A-10C. CadA promotes amoebal plaque formation on dense bacterial lawns. The viability of D. discoideum amoebae was assessed by plating them clonally on growing lawns of K. pneumoniae. (10A) Plaques resulting from equal numbers of AX4 (top) and cadA− mutant (bottom) amoebae plated on K. pneumoniae on SM agar plates (left) or SM agar plates with the nutrient components diluted fivefold (SM/5, right). (10B) Amoebal viability as assessed by plating efficiency of AX4 and cadA− (left y-axis) on K. pneumoniae lawns growing on SM agar plates in which the nutrient components were diluted 1.5- to 5-fold, as indicted. Number of K. pneumoniae cells in representative samples of these platings (red line, right y-axis). Lower and upper bounds of the box plots represent the first and third quartile, respectively, while the whiskers represent maximum and minimum values; the centerline is the median and the “x” represents mean of triplicate samples for three biological replicates (Kruskal-Wallis one-way analysis of variance and ad hoc pairwise Wilcoxon rank sum test); ns, not significant. (10C) Plating efficiency of AX4 and cadA− amoebae on SM agar plates in the presence of exogenous CadA protein (5 μg per spot). Statistical significance was observed between cadA− and cadA− plated with exogenous CadA protein produced in E. coli (rCadA). Box plots represent data from triplicate samples of three biological replicates (Wilcoxon rank sum test, p<0.001).

FIGS. 11A-11B. CadA prevents mixing of bacteria and amoebae across plaque borders. (11A) Representative images of AX4 (upper panels) and cadA− (lower panels) plaques grown on K. pneumoniae on SM/2.5 agar. AX4 plaques form roughly circular plaques with defined edges (upper left panel), while cadA− plaques appear erose with satellite plaques (bottom left panel). No satellite colonies are observed when cadA− amoebae are grown with exogenously added recombinant CadA protein (rCadA). Representative images of H2B-mCherry-expressing AX4 amoebae, showing a defined plaque edge with few cells outside of the plaque, and H2BmCherry-expressing cadA− amoebae migrating away from the plaque edge into the bacterial lawn. The table quantifies the number of cells observed outside of plaques. (11B) Representative images of the edge of AX4 (top) and cadA− (bottom) plaques on K. pneumoniae on SM/2.5 agar, stained with LIVE/DEAD™ BacLight™ stain and visualized by differential interference contrast (DIC) or fluorescence microscopy. SYTO9 (green) labels live bacteria and propidium iodide (PI, red) labels dead bacteria. In the composite images of the plaque edges (far right panels), live bacteria are seen within the growing edge of the cadA− plaque, while the AX4 has predominantly dead bacteria within the border of the plaque.

FIGS. 12A-12C. CadA binds and agglutinates bacteria. (12A) Increasing concentrations of recombinant CadA (1.25-74 μg) were incubated with 10⁹ K. pneumoniae in 600 μl for 60 min. After bacteria were removed by centrifugation, unbound CadA was used to determine the amount of CadA bound to bacteria. Saturation binding of CadA suggests an association constant (Ka) of 1.0×10⁷ M-1 and 7.5×10⁵ binding sites per bacterium. (12B) K. pneumoniae (10⁹) were incubated with recombinant CadA (8 μg) for 60 min., with increasing concentrations (10-300 mM) of D-Glucose (white), D-Glucosamine (black), D-Galactose (grey). Unbound CadA was quantified and used to calculate the amount of CadA bound to the bacteria. Data represent the mean±SD values of three technical replicates of three independent biological replicates (Wilcoxon rank sum test, p<0.001). (12C) K. pneumoniae (10⁹) bacteria were incubated with buffer (upper panels) or recombinant CadA protein (0.66 mg/ml, lower panels), with glucose (middle panels) or galactose (right panels) for 1 hr and visualized by fluorescence microscopy (scale bars=100 μm).

FIGS. 13A-13B. Species-specific restriction of bacterial colony expansion by CadA correlates with CadA bacterial agglutination. (13A) Recombinant CadA (10 μg) or BSA (10 μg) was spotted onto SM agar and allowed to dry. Overnight cultures of K. pneumoniae (upper left panel) and other species of bacteria, as indicated, were spotted adjacent but not touching the CadA spot. Dotted circles represent the original deposition of the proteins and the bars indicate the diameter of the original bacterial spot. (13B) The bacterial species in 13A were incubated with recombinant CadA protein (0.66 mg/ml) and visualized after 1 hr by fluorescence microscopy of the cellpermeable dye SYTO9.

FIGS. 14A-14C. Altered amoebal predation on CadA-agglutinated bacteria. H2B-mCherry-expressing AX4 amoebae (14A), or cadA mutant amoebae (14B), were mixed with K. pneumoniae clumped by CadA (14A, 14B, upper panels) or by mechanically by centrifugation (14A, 14B, lower panels) under agar on glass bottom plates. White arrows highlight amoebae travelling through a clump, while orange arrows highlight amoebae travelling away from a clump. Individual cells were imaged and tracked for 3 hours using Nikon Eclipse Ti (t, minutes; scale bars=100 μm). (14C) Chemotactic index of individual AX4 or cadA− amoebae for clumps of K. pneumoniae that were mechanically produced, CadA-induced, or CadA-induced with 100 μM folic acid added to the buffer prior to plating.

FIGS. 15A-15C. Growth of cadA− on lawns of bacteria. 15A, AX4 and cadA− amoebae were plated with K. pneumoniae and B. subtilis on SM agar and M. luteus and S. aureus on LB agar. Resultant plaques were divided by the number of cells plated. Data represent three biological replicates, each with three technical replicates. The bottom and top of each box plot represent the first and third quartile, respectively, the centerline demarcates the median, the “x” is the mean, and the whiskers are the minimum and maximum observed values. Outliers, defined as 1.5-times the interquartile range, are displayed as red dots. 15B, Diameters of individual amoebal plaques growing on K. pneumoniae were measured from first visualization of each plaque and followed for 4 days. Area was then calculated. Each solid (AX4) and dotted (cadA−) line represent individual plaques and are representative of plaque growth from three biological replicates, each with three technical replicates. 15C, Representative image of cadA− H2b-mCherry plaque growing on a thick lawn K. pneumoniae on SM agar where the white asterisk denotes inset origin (scale bars=100 μm).

FIG. 16. Elevated reactive oxygen species in cadA mutant amoebae feeding on bacteria. Vegetative amoebae were harvested from the edge plaques growing on K. pneumoniae bacterial lawns and stained with CellROX® orange (5 μM) and DAPI for 30 minutes prior to imaging by fluorescence microsocpy.

FIGS. 17A-17C. CadA agglutinates K. pneumoniae and protects them from killing by Dabs. 17A, Protection of K. pneumoniae by CadA from killing by Dictyostelium antibacterial proteins or Dabs (left) and the observed bacterial aggregates within the killing assay (Right). Constant K. pneumoniae were incubated overnight in SM with the described conditions. Inactivation of Dabs by CadA was tested by the protocol outlined. 17B, Increasing amounts of CadA (0.16 mg/ml-1.66 mg/ml) was incubated with 10⁹ GFP-expressing K. pneumoniae. After one hour, cells were washed and imaged. Images are representative of three biological experiments (scale bars=100 μm). Agglutination was quantified by removing aliquots from the top quarter of sample volume after 1 hour and measuring OD₆₀₀. 17C, CadA was incubated with 10 mM EGTA for 60 minutes and then removed by buffer exchange. Following the removal of EGTA, CadA was then incubated with buffer or with 10 mM CaCl₂ before buffer exchange and addition to K. pneumoniae. The treated CadA protein was then added at a concentration of 0.66 mg/ml to K. pneumoniae for one h. Samples were then incubated with LIVE/DEAD™ BacLight™ for 15 minutes. Green fluorescence is cell permeable SYTO9 indicating live bacteria and the red fluorescence is cell impermeable propidium iodide (PI) indicating dead bacteria. Data are representative images from three independent experiments (Scale bars=100 μm).

FIGS. 18A-18B. Differential amoebal movement towards CadA clumped bacteria. AX4 H2b:mCherry (left) and cadA− H2b:mCherry (right) amoebae tracked predating on mechanical, CadA, or CadA with 100 μM folic acid clumped K. pneumoniae GFP. 18A, Speed (μm/minute) was calculated as distance travelled per frame (2 minutes) of individual amoeba. Data is displayed as probability density function of frame-to-frame speed of n>200 amoebae from three independent experiments lasting 390 minutes. 18B, Path length (μm) was calculated as the total path length travelled by individual amoebae. Data represents n=100 amoebae from three independent experiments and is displayed as violin plots with inset box plots. Path length of amoebae predating on CadA clumps is significant from all other conditions (Welsh's t-test, p<0.001). Red points represent outliers as defined as 1.5-times the interquartile range.

### DETAILED DESCRIPTION

In keeping with long-standing patent law convention, the words “a” and “an” when used in the present specification in concert with the word comprising, including the claims, denote “one or more.” Some embodiments of the invention may consist of or consist essentially of one or more elements, method steps, and/or methods of the invention. It is contemplated that any method or composition described herein can be implemented with respect to any other method or composition described herein.

The term “effective amount” as used herein refers to that amount which, when administered to a subject or patient for a clinical purpose, such as to improve gastrointestinal health or other types of health, is sufficient to effect such clinical purpose, including to ameliorate at least one symptom of a gastrointestinal medical condition or to increase the levels of at least one beneficial bacteria and/or to decrease the levels of at least one harmful bacteria.

### I. Examples of Compositions

Compositions of the disclosure include bacteria that externally have one or more types of lectins that are not normally present on the bacteria externally in nature. In specific embodiments, the lectins are bound to the bacteria on the surface of the bacteria. In specific embodiments, the bacteria are coated with one or more types of lectins. In at least some cases, the lectins are present externally on the bacteria at higher or lower density, depending on the number of lectin proteins bound to the surface glycans. In particular embodiments the lectins are bound to their saturation point. They may be present in two or more layers at least in some regions of the bacteria surface. The lectin-bound bacteria are generated by the hand of man and not found in nature. In specific embodiments, the bacteria are one or more types of bacteria produced upon binding of one or more types of lectins to the bacteria.

Embodiments of the disclosure include one or more compositions comprising a plurality of one or more types of isolated bacteria having one or more types of lectins externally associated thereto, and the bacteria may be formulated in a pharmaceutically acceptable carrier, in specific embodiments.

In specific embodiments of the lectin-bound bacteria, the bacteria are nonpathogenic bacteria. The bacteria are live, in particular embodiments. In specific aspects, the bacteria may be lactic acid-producing, although in some cases they are not. In specific embodiments, the bacteria are one or more of Bifidobacterium, Clostridium, Fusobacterium, Melissococcus, Propionibacterium, Streptococcus, Enterococcus, Lactococcus, Staphylococcus, Peptostrepococcus, Bacillus, Pediococcus, Micrococcus, Leuconostoc, Weissella, Aerococcus, Oenococcus, Lactobacillus, Bacteriodetes, Prevotella, Bacteroides, Firmicutes, Xylanibacter, Faecalibacterium, Eubacterium, Subdoligranulum, Parabacteriodetes, Ruminococcus, Collinsella, Rosehburia, Akkermansia, Veillonella, Verrucomicrobia, Escherichia, including a mixture thereof. Particular species of bacteria may include one or more of Bifidobacterium animalis, Escherichia coli, Lactococcus lactis, Lactobacillus reuteri, Tobacillus acidophilus, Acidophilus bifidus, Streptococcus thermophiles, Streptococcus faecium, Clostridium leptum, Clostridium coccoides, Bacillus coagulans, B. lentus, B. licheniformis, B. mesentericus, B. pumilus, B. subtilis, B. natto, Bacteroides amylophilus, Bac. capillosus, Bac. ruminocola, Bac. suis, Bifidobacterium adolescentis, B. animalis, B. breve, B. bifidum, B. infantis, B. lactis, B. longum, B. pseudolongum, B. thermophilum, Candida pintolepesii, Clostridium butyricum, Enterococcus cremoris, E. diacetylactis, E. faecium, E. intermedius, E. lactis, E. muntdii, E. thermophilus, Escherichia coli, Kluyveromyces fragilis, Lactobacillus acidophilus, L. alimentarius, L. amylovorus, L. crispatus, L. brevis, L. case, L. curvatus, L. cellobiosus, L. delbrueckii ss. bulgaricus, L farciminis, L. fermentum, L. gasseri, L. helveticus, L. lactis, L. plantarum, L. johnsonii, L. reuteri, L. rhamnosus, L. sakei, L. salivarius, Leuconostoc mesenteroides, P. cereviseae (damnosus), Pediococcus acidilactici, P. pentosaceus, Propionibacterium freudenreichii, Prop. shermanii, Saccharomyces cereviseae, Staphylococcus carnosus, Staph. xylosus, Streptococcus infantarius, Strep. Salivarius, Strep. thermophilus, Strep. lactis, and a mixture thereof.

In specific embodiments, one or more types of bacteria are coated with one or more types of lectins.

The source of the lectin(s) may be of any kind, including from plants, animals, microorganisms, or a mixture thereof. The lectin(s) may be obtained from its natural source for production of the lectin-bound bacteria, or the lectin may be obtained commercially, or both. The lectin(s) may be of any kind (e.g., C-type, H-type, I-type, L-type, R-type, or Galectins), including at least a mannose-binding lectin, galactose/N-acetylgalactosamine-binding lectin, N-acetylglucosamine-binding lectin, N-acetylneuraminic acid-binding lectin, or Fucose-binding lectin, merely as examples. In certain cases, the lectin is a C-type lectin, L-type lectin, -type lectin, or P-type lectin, for example. Specific examples of lectins include at least Galectin, Concanavalin A, collectin, selectin Lentil lectin, Snowdrop lectin, Ricin, Peanut agglutinin, Jacalin, Hairy vetch lectin, Wheat Germ Agglutinin, Elderberry lectin, Maackia amurensis leukoagglutinin, Maackia amurensis hemoagglutinin, Ulex europaeus agglutinin, Aleuria aurantia lectin, BanLec, Achylectin, Anti-B agglutinin, calnexin, calreticulin, or a mixture thereof.

In particular embodiments, the lectin-bound bacteria are formulated as a probiotic composition. In some cases, the lectin-bound bacteria are formulated as a supplement or a food, for example. The supplement or food may or may not comprise a pharmaceutically acceptable carrier. Embodiments of the disclosure include a probiotic composition that comprise at least one type of lectin-bound bacteria and, in some cases, at least one type of lectin-bound bacteria having at least one type of lectin bound thereto. In certain cases, the probiotic composition comprises more than one type of lectin-bound bacteria each having more than one type of lectin bound thereto. In specific embodiments of a probiotic composition, the composition comprises one of more types of lectin-bound bacteria but also includes one or more other types of bacteria that are not lectin-bound.

Any of the compositions may be in the form of a solid, such as frozen bacteria or in the form of a stab, or they may be in the form of a liquid, such as in culture.

In some embodiments, compositions comprising lectin-bound bacteria for use as a probiotic may be configured to comprise 2, 3, 4, 5, 6, 7, 8, 9, 10, or more genera of bacteria or 2, 3, 4, 5, 6, 7, 8, 9, 10, or more species of bacteria, for example. In specific cases in which the composition comprises multiple types of bacteria, the different bacteria in the composition may or may not be comprised in the composition in a certain ratio. For example, in a composition in which two lectin-bound bacteria are present, the ratio may be 1:1, 1:2, 1:10, 1:25, 1:50, 1:100, 1:500, 1:1000, 1:10000and so forth. In a composition in which three bacteria are present, the ratio may be 1:2:1, 1:5:1, 1:10:1, 1:100:1, 1:1000:1, 2:2:1, 2:5:1, 2:10:1, 2:100:1, 2:1000:1, 1:2:2, 1:2:5, 1:2:10, 1:2:50, 1:2:100, 1:2:1000, and so forth.

In a composition comprising lectin-bound bacteria, the concentration of bacteria in the composition may be of any suitable amount. In specific embodiments, the bacteria are present in the composition at a concentration in a range of 10³ bacteria/ml to 10¹² bacteria/ml. In specific embodiments, the concentration is in a range of 10³ to 10¹², 10⁴ to 10¹², 10⁵ to 10¹², 10⁶ to 10¹², 10⁷ to 10¹², 10⁸ to 10¹², 10⁹ to 10¹², 10¹⁰ to 10¹², 10¹¹ to 10¹², 10³ to 10¹¹, 10⁴ to 10¹¹, 10⁵ to 10¹¹, 10⁶ to 10¹¹, 10⁷ to 10¹¹, 10⁸ to 10¹¹, 10⁹ to 10¹¹, 10¹⁰ to 10¹¹, 10³ to 10¹⁰, 10⁴ to 10¹⁰, 10⁵ to 10¹⁰, 10⁶ to 10¹⁰, 10⁷ to 10¹⁰, 10⁸ to 10¹⁰, 10⁹ to 10¹⁰, 10³ to 10⁹, 10⁴ to 10⁹, 10⁵ to 10⁹, 10⁶ to 10⁹, 10⁷ to 10⁹, 10⁸ to 10⁹, 10³ to 10⁸, 10⁴ to 10⁸, 10⁵ to 10⁸, 10⁶ to 10⁸, 10⁷ to 10⁸, 10³ to 10⁷, 10⁴ to 10⁶, 10⁵ to 10⁶, 10³ to 10⁵, 10⁴ to 10⁵, or 10³ to 10⁴ bacteria/ml.

### II. Examples of Methods of Use

The disclosure encompasses a variety of uses for lectin-bound bacteria. Methods include those for beneficially altering a microbiome in a subject. Such methods include those for improving a microbiome in a subject compared to the state of the microbiome in the absence of use of the lectin-bound bacteria. In specific embodiments, the methods encompass use of lectin-bound bacteria to enhance retention of non-pathogenic bacteria in a microbiome, including beneficial non-pathogenic bacteria, in any microbiome. Such retention includes maintaining a level of one or more particular bacteria in a microbiome and also includes maintaining a substantially steady state level of one or more particular bacteria in a microbiome.

In some embodiments, a composition comprising certain lectin-bound bacteria are employed to modify a microbiome by increasing the levels of bacteria that were previously in the microbiome, whereas in other cases certain lectin-bound bacteria are employed to modify a microbiome by increasing the levels of bacteria that were not previously in the microbiome (or were not at significant levels in the microbiome). Thus, in some cases the compositions provide new type of bacteria that have analogous benefits to the microbiome as an existing type of bacteria in the microbiome.

Methods of the disclosure include methods of modulating one or more microbiomes. The modulating step may be performed for the purpose of introducing one or more beneficial bacteria into an existing microbiome that may or may not have previously comprised the particular one or more beneficial bacteria. In some methods the modulating step is performed for the purpose of increasing the level of one or more beneficial bacteria in a microbiome that previously or currently comprised the one or more beneficial bacteria. Increasing the level of one or more bacteria in a microbiome may or may not include raising a level in a microbiome to previous levels or to higher levels than a previous level.

The methods of the disclosure may concern modification of particular microbiomes for health benefit of one or more subjects or for agricultural benefit for one or more subjects. The methods may be employed to modulate, including improve, the microbiome of one or more types of subject(s) wherein the subject(s) may be an animal. The animal may be used for consumption by others or for production of materials (for example, for their hide, muscle, organs, etc.). The subject whose microbiome is modulated may be a mammal or non-mammal. The subject may be a farm animal or livestock animal. The animal may be an animal hunted for consumption or otherwise used for sport (for example, ungulates). In cases wherein the subject is a mammal, the mammal may be a human, dog, cat, horse, lamb, sheep, pig, goat, rabbit, or cow. The subject may be a fish (in which case the microbiome may be present in the swim bladder) or bird (such as chickens or turkeys). Any subject may be of any gender or age, including infant, child, adolescent, or adult, including elderly adults (for example, over the age of 65). A subject may begin use of the lectin-bound bacteria upon onset of a medical condition or as part of routine health maintenance or upon being of a certain age, for example.

In specific embodiments, the subject in need of microbiome modification is a plant, including an agricultural plant. Examples of agricultural plants include any type of crop, such as corn, soybean, rice, sorghum, sweet potato, yams, wheat, barley, cassava, cotton, peanut, millet, potato, plaintains, oats, tobacco, and so forth. It also includes olive trees, vegetable plants (including cucumbers, peas, beans, lentils, tomatoes, eggplants, bell peppers, cauliflower, cabbage, brussels sprouts, broccoli, onions, garlic, leek, shallot, chives, carrots and lettuce), and so forth. It also includes herbs of any kind and all citrus-bearing plants, such as orange trees, lemon trees, lime trees, grapefruit trees, etc. The plant may be an ornamental plant, such as garden plants (flowering (such as roses; ornamental bulb plants; conifers; ornamental grasses; ornamental shrubs; and so forth) and non-flowering) and trees.

Methods of the disclosure include methods of pre-treating one or more types of bacteria with one or more types of lectins to provide a protein covering, such as a coat. Particular methods encompass coating of bacteria with lectins to protect them from destruction by one or more antibacterial agents, including one or more antibacterial proteins, for example. Such protection methods may occur outside cells, for example; the protection methods may occur in vivo, ex vivo, or in vitro.

Particular methods of the disclosure may limit the growth of one or more types of harmful (for example, pathogenic) bacteria in the microbiome, and such limiting of growth may be the purpose of the method or may be a direct or an indirect benefit of the method. Examples of harmful bacteria include Clostridium difficile, for example. In some cases, the introduction of the lectin-bound bacteria in the microbiome increases the overall level of nonpathogenic bacteria compared to pathogenic bacteria, and such an increase is beneficial to the subject (in some cases, regardless of the type(s) of lectin-bound bacteria).

Methods of the disclosure include methods of preparing bacteria to be impervious or less susceptible to one or more harmful agents to the bacteria, such as one or more harmful microorganisms and/or one or more antibacterial agents (including proteins) and/or one or more environmental conditions, such as acidic conditions. In some cases, the type of lectin utilized for the lectin-bound bacteria determines the effectiveness of the lectin to protect the bacteria from one or more harmful microorganisms, agents, and/or conditions.

Formulations of lectin-bound bacteria may be utilized in methods of improving the retention of one or more types of beneficial bacteria, and in some cases such methods encompass improving the uptake by an organism of one or more beneficial bacteria. Methods of the disclosure provide for more efficient use of beneficial bacteria.

Methods of the disclosure may be implemented in individuals that are generally healthy, such as individuals that do not have a gastrointestinal medical condition (for example, irritable bowel syndrome; inflammatory bowel disease (IBD); infectious diarrhea (caused by viruses, bacteria, or parasites); or diarrhea caused by antibiotics), for example. The methods may be implemented in individuals that do have a gastrointestinal medical condition or they may be at risk for a gastrointestinal medical condition, such as at risk compared to the general population. The individual at risk may have a family and/or personal history and/or may have one or more risk factors, such as one or more biological or genetic markers that predispose them to a gastrointestinal medical condition.

The subject may be determined by a medical practitioner to be in need of probiotic(s), such as having one or more symptoms of a gastrointestinal medical condition. In other cases, methods may be implemented for an individual as a part of routine good health practices. In specific embodiments, an individual begins taking the lectin-bound bacteria upon the onset of at least one symptom of a gastrointestinal medical disorder, such as acid reflux, heartburn, dyspepsia/indigestion, nausea and/or vomiting, belching, bloating, and/or flatulence, for example.

In particular cases, a subject will be provided (such as for ingesting) the lectin-bound bacteria as a single dose, although in some cases the subject will be provided lectin-bound bacteria in multiple doses. In cases wherein there are multiple doses to be taken, it may be once daily; more than once daily, such as 2 or 3 times daily; weekly; more than once weekly, such as 2, 3, 4, 5, 6 or more times weekly; monthly; more than once monthly, such as 2, 3, 4, 5, 6, or more times monthly, and so forth. In some cases the subject may be asymptomatic at the onset of the treatment, whereas in other cases the subject may have one or more symptoms (such as having one or more of acid reflux, heartburn, dyspepsia/indigestion, nausea and/or vomiting, belching, bloating, and/or flatulence). The subject may continue taking the lectin-bound bacteria after all or most symptoms are gone and for some period of time, such as for days, weeks, or months.

In some cases, upon administration of the lectin-bound bacteria, one or more symptoms may be reduced entirely, reduced in intensity, and/or delayed in the timing of onset.

In some embodiments, a subject exposed to one or more methods of the disclosure may consume one or more prebiotics to enhance the efficacy of the probiotic methods of the disclosure. In specific embodiments, the subject consumes one or more foods that support increased levels of SCFA, such as indigestible carbohydrates and fibers including inulin, resistant starches, gums, pectins, and/or fructooligosaccharides. The prebiotics may be supplements and/or may be foods naturally containing prebiotics. Examples of such foods include at least garlic, nions, leeks, asparagus, Jerusalem artichokes, dandelion greens, bananas, and seaweed, for example. In general, fruits, vegetables, beans, and whole grains like wheat, oats, and barley may be utilized as sources of prebiotic fibers.

In some embodiments, a subject exposed to one or more methods of the disclosure may consume one or more probiotics in addition the probiotic compositions encompassed herein. Examples include probiotic foods, such as probiotic foods that comprise beneficial live microbiota including fermented foods like kefir, yogurt with live active cultures, pickled vegetables, tempeh, kombucha tea, kimchi, miso, and sauerkraut, for example.

### III. Examples of Methods of Producing the Bacteria

In general embodiments, lectin-bound bacteria are produced prior to their use by one or more subjects. The party generating the lectin-bound bacteria may or may not be the party delivering the lectin-bound bacteria to the subject. The bacteria may be produced and utilized immediately, or they may be stored.

In general embodiments, the lectin-bound bacteria are produced by exposing a sufficient amount of one or more types of lectin(s) to a sufficient amount of one or more types of bacteria. The production methods may or may not be such that the bacteria have lectin bound thereto in the form of a coat. In cases wherein the lectin are being bound to the bacteria in the form of a coat, the coating of lectin may or may not be one or more layers in thickness. Conditions may be determined using routine steps by the skilled artisan as to the amount of lectin(s) and bacteria and the duration of the binding steps for suitable production.

Following production of the lectin-bound bacteria, the bacteria may be used directly or they may be stored. In specific embodiments the lectin-bound bacteria are transported before or after storage. The lectin-bound bacteria may be frozen, in specific embodiments.

In some cases, the production of the lectin-bound bacteria encompasses a selection step of selecting one or more types of lectin to be bound to the selected one or more types of bacteria. For a particular purpose, such as modifying a microbiome in a subject having a gastrointestinal medical condition, one or more particular types of bacteria may be selected for the subject and also particular one or more types of lectins may be selected for binding to the one or more particular types of bacteria. In another example, a microbiome of an agricultural plant may be modified with selection of one or more particular types of bacteria and with one or more particular types of lectins. One or more types of bacteria can be produced by standard industrial fermentation processes to produce sufficient amounts (for example, pounds) of the desired bacteria. Lectin proteins could be obtained from commercial sources or purified from the source organism using industrial-scale biochemical purification technologies.

### IV. Examples of Methods of Screening

In some embodiments of the disclosure, there are methods of screening for a suitable one or more types of lectins for a particular one or more types of bacteria. In specific embodiments, one or more particular types of bacteria are determined to be suitable and desired for a subject. Following this, there is determination of a suitable one or more types of lectins to be bound to the selected bacteria.

In some cases, the bacteria are screened for the ability to bind one or more types of lectins. The methods may incorporate an assay that provides a detectable output providing information as to the ability of lectin(s) to bind and the efficacy of the lectin(s) in protecting the bacteria. Such a method may or may not be high throughput, in specific embodiments. Standard solution assays may be used to assess lectin binding to any bacterium of interest, for individual lectins or combinations of two or more lectins. When the desired lectin binding is identified, the lectin-bound bacteria may be tested for resistance to various anti-bacterial agents, including antibacterial proteins (e.g., pore-forming peptides, lysozyme, and/or hydrolytic enzymes, such as proteases), antibiotics, or environmental conditions (e.g., temperature, pH, and/or high or low ionic conditions). Such tests may inform which lectin is most useful for protecting the bacteria for specific applications.

V. Examples of Methods of Incorporation of Bacteria into Cells

Embodiments of the disclosure include methods of facilitating uptake of bacteria into a cell or cells. Although the cell may be of any kind, in specific embodiments the cell is a mammalian cell, such as a human cell. Examples of mammalian cells include at least epithelial cells, cancer cells, immune system cells, primary cell, established cell line, hybridoma cell, germ cell, somatic cell, stem cell, progenitor cell, pluripotent cell, totipotent cell, or a mixture thereof. The mammalian cell may be from the ectoderm, mesoderm, or endoderm.

The method may occur ex vivo, in vivo, or in vitro and may comprise the step of binding one or more lectins to the bacteria in a sufficient amount for the bacteria to be uptaken into the cell. The lectin may be derived from one or more plants, amoeba, animals, or a mixture thereof. The bacteria may be Gram-negative or Gram-positive. The bacteria may be pathogenic or non-pathogenic. The bacteria may be spherical, rod shaped, or spiral. Examples of specific bacteria include the type Coccus, Bacillus, Vibrio, Spirillum, or Spirochete. The bacteria may be genetically engineered, such as comprising one or more man-made mutations or exogenous DNA element designed to express a specific exogenous protein or RNA.

In such methods, the lectin may be mannose-binding, Galactose/N-acetylgalactosamine-binding, N-acetylglucosamine-binding, N-acetylneuraminic acid-binding, or fucose-binding, for example.

Although the bacteria may be of any kind, in specific embodiments the bacteria is E. coli.

In some embodiments, the method of incorporation of lectin-bound bacteria into one or more types of cells is for a therapeutic application, a manufacturing application, or a research application, for example delivering a small molecule, RNA molecule, DNA molecule or combination thereof.

### VI. Pharmaceutical Preparations

Pharmaceutical compositions of the present disclosure comprise an effective amount of one or more types of lectin-bound bacteria dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one lectin-bound bacteria will be known to those of skill in the art in light of the present disclosure, as exemplified by Remington: The Science and Practice of Pharmacy, 21^{st} Ed. Lippincott Williams and Wilkins, 2005, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The lectin-bound bacteria may comprise different types of carriers depending on whether it is to be administered in solid, liquid or aerosol form, and whether it need to be sterile for such routes of administration as injection. Although in particular embodiments the lectin-bound bacteria are administered enterically, including at least orally, although the present bacteria in alternative embodiments can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference). In specific embodiments, the lectin-bound bacteria are administered at one or more points along the alimentary canal of the subject.

The lectin-bound bacteria may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present disclosure, the composition of the present disclosure suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present disclosure, the lectin-bound bacteria composition is combined with the carrier in any convenient and practical manner, i.e., by solution, suspension, emulsification, admixture, encapsulation, absorption and the like. Such procedures are routine for those skilled in the art.

In a specific embodiment of the present disclosure, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present disclosure may concern the use of a pharmaceutical lipid vehicle compositions that include lectin-bound bacteria, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds that contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the lectin-bound bacteria may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present disclosure administered to an animal patient can be determined by physical and physiological factors such as body weight, severity of condition, the type of disease being treated, previous or concurrent therapeutic interventions, idiopathy of the subject and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 mg to 1 g dry weight. In specific embodiments the dose is from about 1 mg to 750 mg, 1 mg to 500 mg, 1 mg to 250 mg, 1 mg to 100 mg, 50 mg to 1 g, 50 mg to 750 mg, 50 to 500 mg, 50 to 250 mg, 50 to 100 mg, 100 mg to 1 g, 100 mg to 750 mg, 100 mg to 500 mg, 100 mg to 250 mg, 250 mg to 1 g, 250 mg to 750 mg, 250 mg to 500 mg, 500 mg to 1 g, 500 mg to 750 mg, and so forth.

In particular embodiments, the lectin-bound bacteria are formulated in alimentary compositions and formulations. In particular embodiments of the present disclosure, the lectin-bound bacteria are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792, 451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration, the compositions of the present disclosure may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations that are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

### EXAMPLES

The following examples are included to demonstrate particular embodiments of the disclosure. It should be appreciated by those of skill in the art that the techniques disclosed in the examples that follow represent techniques discovered by the inventors to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Lectins Modulate the Microbiota of Social Amoebae

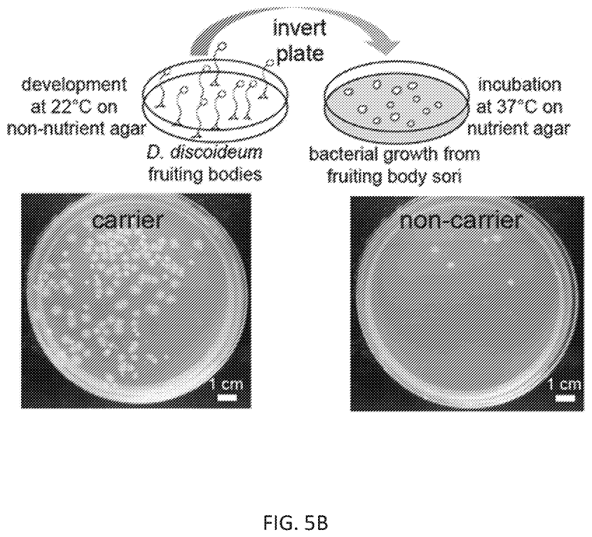

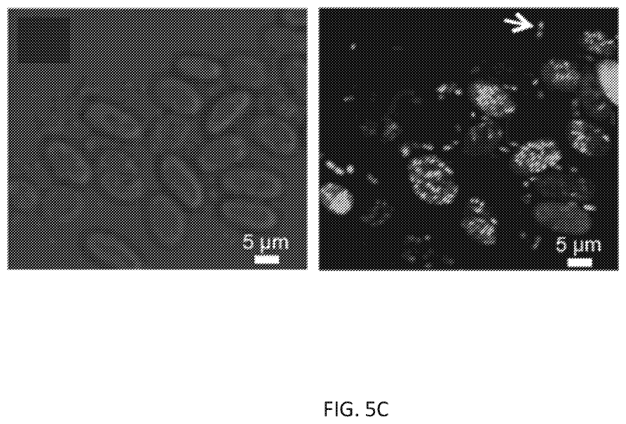

Carriers are easily distinguished from non-carriers because migrating carrier slugs leave behind bacteria that form colonies visible to the naked eye (FIG. 5A). To assay for carriage, we allow amoebae to initiate development in the presence of food bacteria such as Klebsiella pneumoniae (FIG. 5B) and score individual fruiting bodies for the presence of viable bacteria (FIG. 5C). By this measure >90% of a carrier strain's sori contain bacteria (Table 1) and bacterial endosymbionts in their spores (FIG. 5D), while <10% of sori from non-carrier strains contain any viable bacteria (Table 1).

TABLE 1
Bacterial carriage by wild isolates of
D. discoideum and induced by lectin

| | | | sori with live bacteria (%)⁴ | | |
|---|---|---|---|---|---|
| | bacterial | | fruiting | | fruiting after |
| | carriage | Sentinel cells | body | fruiting | exposure to |
| | pheno- | (% of | development | after slug | lectin-coated |
| Strain | type² | slug cells)³ | in situ | migration⁵ | bacteria |
| AX4¹ | car⁻ | — | 2.5 ± 2.0 | <3⁶ | 100⁷ |
| QS4 | car⁻ | 1.0 ± 0.04 | 4.3 ± 1.2 | <3⁶ | 36 ± 8 |
| QS34 | car⁻ | 1.0 ± 0.40 | 8.3 ± 2.1 | <3⁶ | — |
| QS40 | car⁻ | 1.6 ± 0.14 | 8.0 ± 3.8 | <3⁶ | — |
| QS37 | car⁺ | 0.40 ± 0.02 | 95 ± 3.5 | 53 ± 8.4 | — |
| QS73 | car⁺ | 0.30 ± 0.25 | 97 ± 2.0 | 83 ± 5.3 | — |
| QS74 | car⁺ | 0.15 ± 0.12 | 99 ± 1.4 | 84 ± 9.6 | — |

¹Laboratory strain that has been extensively characterized (12, 13).
²Determined by the presence/absence of bacteria in slime trails of migrating slugs (e.g., FIG. 5A).
³Mean percentage (±s.e.m.) determined by flow cytometry.
⁴Mean percentage (±s.e.m.) of sori with cultivatable bacteria.
⁵Sori from fruiting bodies that had formed from slugs after they had migrated for 6 cm.
⁶No bacterial outgrowth from any of 30 sori tested in each of 3 experiments.
⁷Bacterial outgrowth from all sori tested in each of 3 independent experiments.

It is unclear how bacterial carriage occurs given the existence of an innate immune system though significantly reduced Sentinel cell populations in carrier strains has been reported (9). All of our carrier strains have Sentinel cells (Table 1), so we tested whether they cleared bacteria during extended slug migration. We found that carriers maintained their bacteria through >6 cm of slug migration, while no viable bacteria were recovered from non-carriers after slug migration, suggesting that S cells do not influence bacterial carriage (Table 1).

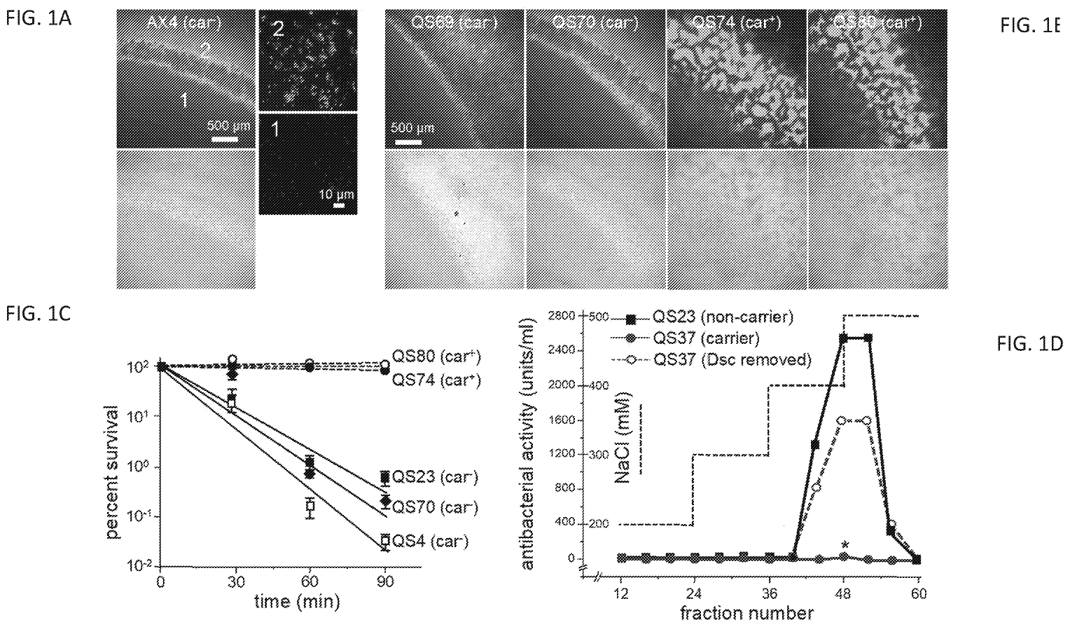

Since carrier strains spare bacteria for development, we assessed their ability to kill bacteria as they transitioned from growth to development. The non-carrier laboratory strain AX4 kills extracellular bacteria within the plaque, while just outside the plaque the bacteria are alive (FIG. 1A). Plaques of non-carrier strains were similar to AX4's, whereas plaques of carriers had a mottled appearance with live and dead bacteria segregated into distinct areas near the edges (FIG. 1B). Thus, extracellular bacterial killing within carrier plaques is uneven and this might explain the presence of live bacteria as the amoebae begin to develop.

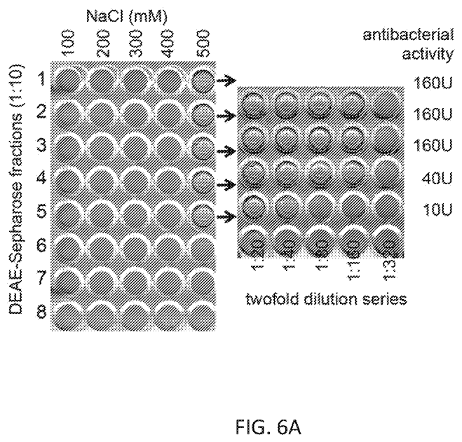

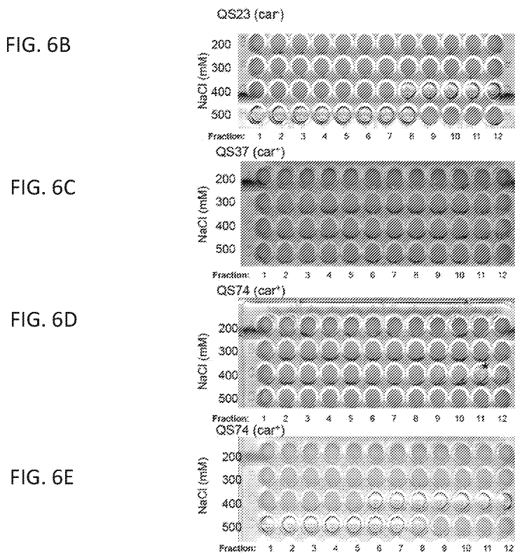

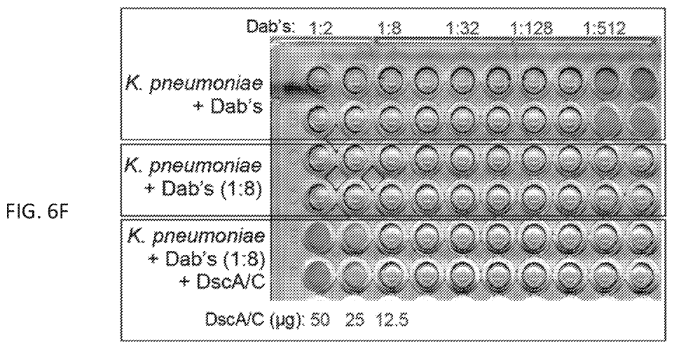

We tested extracellular killing of K. pneumoniae by amoebae, in suspension, and found that non-carrier strains killed bacteria efficiently, but carrier strains did not (FIG. 1C). We tested whether this difference in bacterial killing might be explained the proteins they secrete during growth by examining the secretomes harvested from amoebae consuming K. pneumoniae. We fractionated the secretomes by ion-exchange chromatography and followed antibacterial activity using an end-point dilution assay that measures the killing of K. pneumoniae (FIG. 6A). Antibacterial activity in non-carrier secretomes eluted from the column in a broad peak and was heat-labile (10 min, 65° C.), but carriers produced very little, or undetectable, activity (FIG. 1D; FIG. 6B-6D; Table 2).

TABLE 2
Antibacterial activity of secreted proteins from D. discoideum.

| | crude | | Sepharose flow-through |
|---|---|---|---|
| strain or protein | secretome² | DEAE peak³ | DEAE peak⁴ |
| AX4 (car⁻) | 4.7 × 10² | 1.3 × 10⁴ | — |
| QS4 (car⁻) | 5.3 × 10² | 1.1 × 10⁴ | — |
| QS23 (car⁻) | 5.8 × 10² | 1.1 × 10⁴ | — |
| QS37 (car⁺) | <62 | <1.6 × 10³ | 1.1 × 10⁴ |
| QS73 (car⁺) | <95 | <2.0 × 10³ | 8.9 × 10³ |
| QS74 (car⁺) | <63 | <1.2 × 10³ | 8.3 × 10³ |

¹Results of typical assays for representative purification experiments are shown.
²The indicated D. discoideum strain was mixed with K. pneumoniae for 2 hours and the suspension was clarified by centrifugation and antibacterial activity determined by an end-point dilution K. pneumoniae killing assay (FIG. 6A). No activity was detected in secretomes from car⁺ strains so an upper activity limit is shown.
³Peak fractions eluted from a DEAE column (e.g., FIG. 1D) were pooled and assayed (FIG. 6A). No activity was detected in any DEAE fractions from car⁺ strains so an upper activity limit is shown.
⁴Carrier secretomes were first passed over a Sepharose CL4B column and the flow-through from that column was loaded onto a DEAE column and batch-eluted with NaCl.

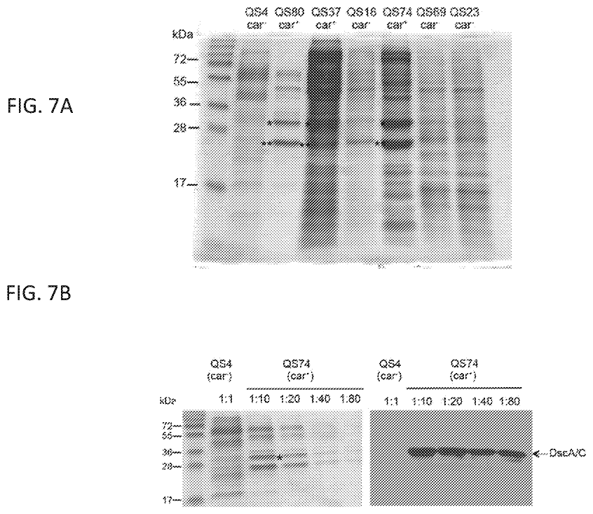

This prompted us to identify regulators of bacterial carriage by screening for proteins that are differentially secreted by carrier versus non-carrier amoebae. The most prominent differences between carriers and non-carriers were two proteins with apparent molecular weights of ˜24 kDa and ˜30 kDa (FIG. 7A). We used mass spectrometry to determine that the 30-kDa band contained a mixture of the well-studied discoidin I lectins discoidin A (DscA) and discoidin C (DscC) (Table 3) and the 24-kDa band contained a mixture of the calcium dependent adhesion protein CadA and the related proteins Cad2 and Cad3 (Table 4)(10-13).

TABLE 3
Identification of discoidin I protein in the
secretome of a carrier strain by mass spectrometry.
Strain QS74 - 30 kDa band Top 30 protein I.D.s

| Unique | Total | | | MW |
|---|---|---|---|---|
| Peptides | Peptides | Reference | Gene Name | kDa |
| 21 | 451 | DIS1A_DICDI | dscA | 28.24 |
| 9 | 174 | DIS1B_DICDI | dscC | 28.37 |
| 19 | 60 | 1433_DICDI | fttB | 28.69 |
| 18 | 51 | CISYM_DICDI | cs | 51.18 |
| 8 | 44 | IDHB_DICDI | idhB | 38.66 |
| 14 | 26 | Q54X02_DICDI | DDB_0205702 | 52.63 |
| 15 | 24 | ARPC2_DICDI | arcB | 32.91 |
| 5 | 23 | Q54F16_DICDI | DDB_0219654 | 38.57 |
| 10 | 22 | ACT10_DICDI | act10 | 41.72 |
| 20 | 21 | Q55CE1_DICDI | DDB_0201947 | 84.84 |
| 17 | 19 | HPPD_DICDI | hpd | 41.71 |
| 14 | 18 | GBLP_DICDI | gpbB | 36.21 |
| 14 | 14 | ADT_DICDI | ancA | 33.45 |
| 11 | 13 | RL8_DICDI | rpl8 | 27.5 |
| 12 | 12 | RS3A_DICDI | rps3a | 30.34 |
| 11 | 12 | SMT1_DICDI | smt1 | 39.53 |
| 11 | 11 | RL5_DICDI | rpl5 | 32.78 |
| 10 | 11 | PSMD2_DICDI | psmD2 | 98.3 |
| 10 | 11 | RS4_DICDI | rps4 | 30.02 |
| 10 | 10 | MCFN_DICDI | mcfN | 31.67 |
| 9 | 9 | RS6_DICDI | rps6 | 26.15 |
| 6 | 8 | Q54KZ1_DICDI | DDB_0187236 | 38.6 |
| 7 | 7 | METE_DICDI | metE | 94.46 |
| 6 | 6 | ANXA7_DICDI | nxnA | 51.14 |
| 6 | 6 | BIP2_DICDI | bip2 | 72.49 |
| 6 | 6 | CAD1_DICDI | cadA | 23.91 |
| 6 | 6 | PGK_DICDI | pgkA | 45.68 |
| 6 | 6 | RL10A_DICDI | rpl10a | 24.32 |
| 5 | 6 | EF2_DICDI | efbA | 92.6 |
| 5 | 5 | AMPL_DICDI | lap | 56.37 |

TABLE 4
Identification of CadA protein in the secretome of
carrier strains by mass spectrometry.
Strain QS74 - 24-kDa band Top 30 protein I.D.s

| Unique | Total | | | MW |
|---|---|---|---|---|
| Peptides | Peptides | Reference | Gene Name | (kDa) |
| 24 | 136 | CAD1_DICDI | cadA | 23.91 |
| 16 | 89 | CAD2_DICDI | cad2 | 22.5 |
| 19 | 83 | CAD3_DICDI | cad3 | 23.59 |
| 12 | 43 | OAT_DICDI | oatA | 45.72 |
| 9 | 35 | ACT10_DICDI | act10 | 41.72 |
| 21 | 30 | MDHB_DICDI | mdhB | 37.62 |
| 19 | 23 | ARGE_DICDI | argE | 49.04 |
| 10 | 21 | PSB4_DICDI | psmB4-1 | 28.99 |
| 14 | 19 | DNPEP_DICDI | dnpep | 53.32 |
| 17 | 18 | Q55CE1_DICDI | DDB_0201947 | 84.84 |
| 15 | 18 | Q54Q52_DICDI | DDB_0185839 | 26.36 |
| | | | DDB_G027682 | |
| 13 | 17 | AL7A1_DICDI | 1 | 55.16 |
| 14 | 15 | Q9BI24_DICDI | 3B | 24.26 |
| 13 | 13 | PSA2_DICDI | psmA2 | 25.65 |
| 12 | 13 | PSA7_DICDI | psmA7 | 27.62 |
| 11 | 13 | PSB1_DICDI | psmB1 | 26.36 |
| 11 | 13 | Q54QK0_DICDI | DDB_0185683 | 22.09 |
| 10 | 12 | GSTA3_DICDI | gsta3 | 23.59 |
| 9 | 11 | NDKC_DICDI | ndkC-1 | 16.78 |
| 10 | 10 | GELA_DICDI | abpC | 92.15 |
| | | | DDB_G028391 | |
| 9 | 10 | Q54QE7_DICDI | 5 | 25.96 |
| 9 | 10 | PRDX4_DICDI | prdx4 | 29.02 |
| 9 | 9 | Q86IL9_DICDI | DDB_0168714 | 31.01 |
| 9 | 9 | HUTH_DICDI | halDDB_G027189 | 58.91 |
| 8 | 9 | Q869Z5_DICDI | 2 | 26.25 |
| 8 | 9 | PSA6_DICDI | psmA6 | 27.2 |
| 8 | 9 | PSB7_DICDI | psmB7 | 28.64 |
| 8 | 8 | SODM_DICDI | sod2 | 25.11 |
| 8 | 8 | Q54KZ1_DICDI | DDB_0187236 | 38.6 |
| 7 | 8 | DIS1A_DICDI | dscA-1 | 28.24 |

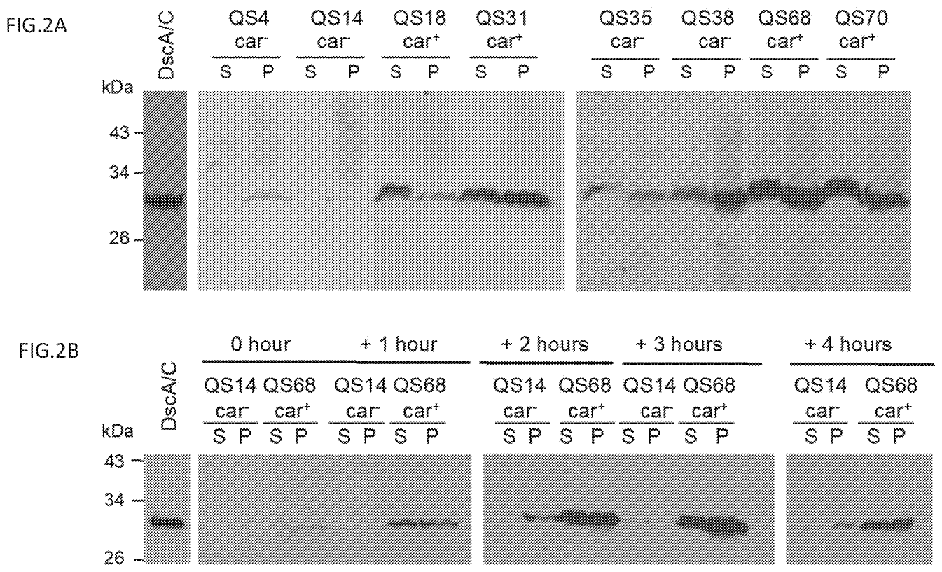

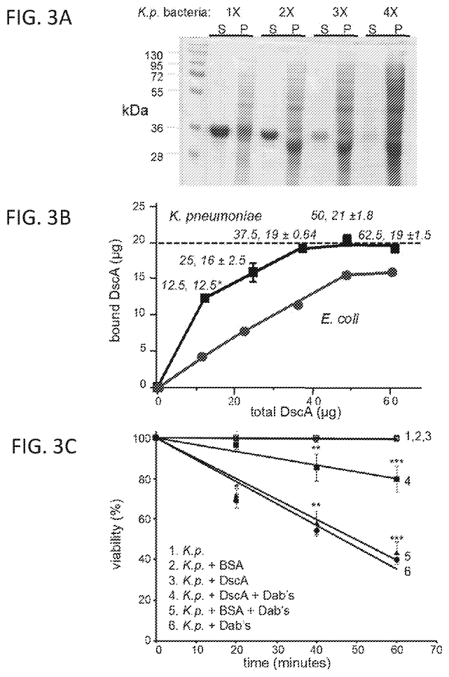

Discoidin I forms dumbbell-shaped trimers with the subunits oriented in parallel, positioning the C-terminal H-type lectin domains at one end (14). Discoidin I binds to polysaccharides containing GalNAc and galactosamine in β1-3 linkages that are found in D. discoideum cell surface glycoproteins and bacterial carbohydrates (15, 16). In laboratory strains, discoidin I is found inside amoebae and it is secreted when they aggregate after the start of development (17, 18). We surveyed the secretion of discoidin I in wild strains, using antibodies against DscA and DscC, and carriers appeared to secret higher levels of these proteins at the start of development compared to non-carriers (FIG. 2A). One carrier that we examined in detail secreted >80-times more discoidin I than non-carriers (FIG. 7B). We examined the timing of discoidin I secretion from the end of vegetative growth through developmental aggregation and found that carriers secreted higher amounts of discoidin I at earlier times compared to non-carriers (e.g., FIG. 2B). These results suggest that discoidin I secretion prior to development is a physiological feature of carriers that may determine bacterial carriage. To explore this we first measured bacterial binding and found that DscA binds quantitatively to K. pneumoniae bacteria, likely through the GalNAc moieties present in the lipopolysaccharide of many K. pneumoniae strains (FIG. 3A)(14, 19). DscA displayed saturation binding to K. pneumoniae and E. coli (FIG. 3B), and we estimated that DscA binds to K. pneumoniae with micromolar affinity (Kₐ=1.2×10⁶ M⁻¹) through ˜1.4×10⁶ binding sites on each bacterium (20). Approximately 2.75×10⁵ DscA proteins would be required to form a monolayer covering the surface of one K. pneumoniae bacterium, assuming that the lectin ends of the roughly cylindrical trimers were opposing an idealized smooth surface with the shape and volume of the bacterium. Thus, additional binding sites within the glycocalyx of K. pneumoniae likely account for the fivefold more DscA binding that we observe.

We tested whether discoidin I binding alters K. pneumoniae's interaction with amoebae, by examining bacterial killing, carriage and endosymbiosis. First, we tested whether discoidin I protects bacteria from killing by D. discoideum antibacterial proteins (Dab's). Indeed, DscA-bound K. pneumoniae were protected from killing by Dab's in an overnight outgrowth assay (FIG. 6F), and in a short-term viability assay (FIG. 3C). Significantly, the protection in the outgrowth assay diminished as the amount of discoidin added was below that needed to achieve saturation binding to the bacteria in the assay (FIG. 6F). However, DscA-coated K. pneumoniae were not resistant to killing by heat (65° C., 10 min) or antibiotic treatment (kanamycin at 50 μg/ml). If protection by discoidin I is a mechanism by which carrier strains spare bacteria, carriers would have to secrete sufficient discoidin to protect some bacteria as the amoebae enter development. Thus, we re-examined carrier secretomes for cryptic Dab's that might have been masked by discoidin I. Indeed, removing discoidin I from carrier secretomes by passing them through poly-galactose affinity columns revealed latent antibacterial activity (FIG. 1D, FIG. 6E; Table 2). Our results indicate that discoidin I can counteract the Dab's secreted by carriers, at least in vitro, and this suggests that discoidin I is secreted by carrier strains at levels sufficient to spare some bacteria for carriage as amoebae enter development.

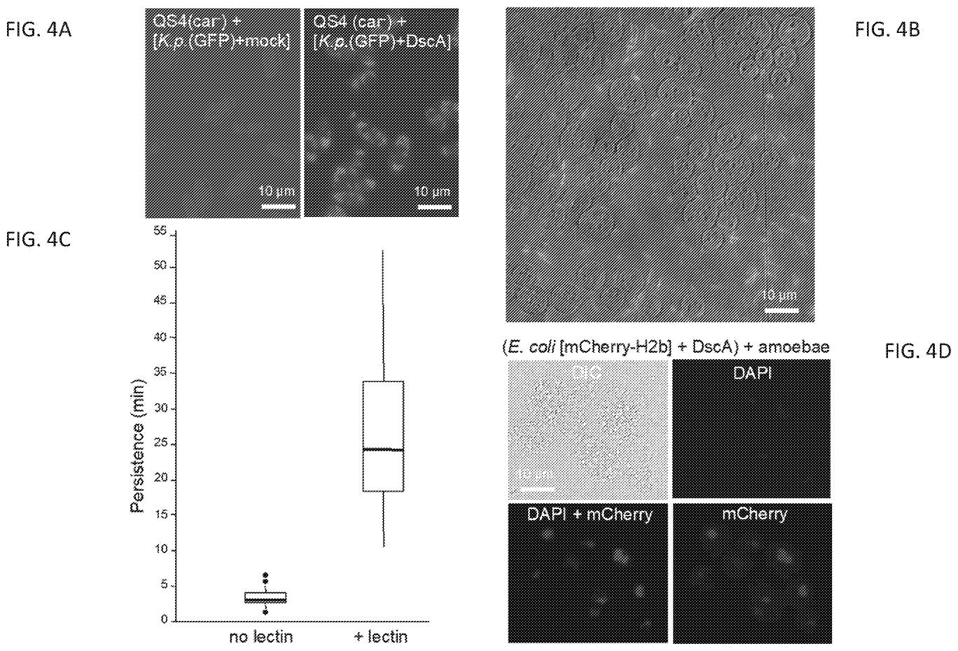

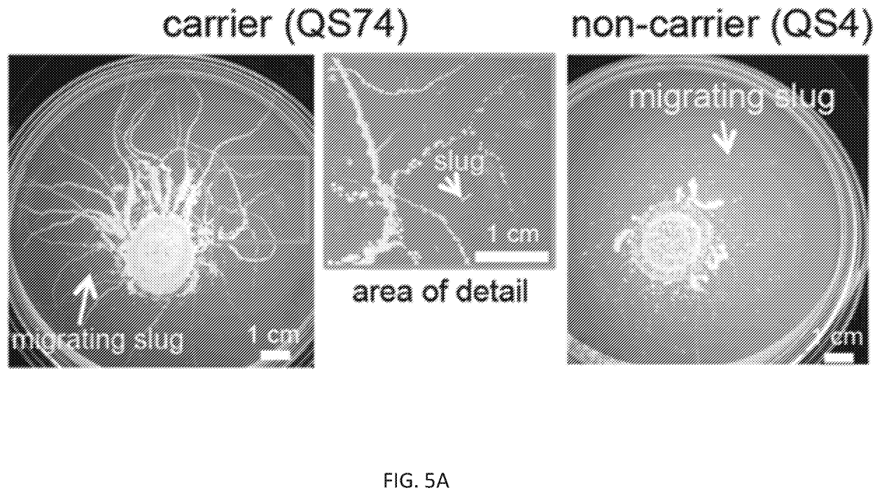

We tested whether discoidin I influences the bacterial carriage and endosymbiosis that defines the carrier phenotype. We carried out the standard test for bacterial carriage by mixing D. discoideum amoebae with K. pneumoniae, or with DscA-coated K. pneumonia at the start of development and tested the resulting fruiting body sori for live bacteria. A single treatment of K. pneumoniae with DscA resulted in bacterial carriage by the non-carrier strain QS4 and the non-carrier laboratory strain AX4 (Table 1). We also observed live green fluorescent protein (GFP) expressing K. pneumoniae within the D. discoideum spores, but only when the bacteria were pretreated with DscA (FIG. 4A). These results suggest that extracellular discoidin I is sufficient to account for bacterial carriage in D. discoideum by binding bacteria, protecting them from secreted antibacterial proteins, and inducing carriage and endosymbiosis.

Some of the internalized discoidin-coated bacteria escape intracellular killing and digestion by the phagolysosomal pathway of the amoebae and end up the spores. Thus, discoidin I alters the handling of bacteria by amoebae such that live bacteria persist within amoebae, a phenomenon we have termed lectin-induced modified bacterial internalization, or LIMBI. We demonstrated the specificity of LIMBI for lectin-coated bacteria with mixing experiments of discoidin-coated bacteria and uncoated bacteria. We presented DscA-bound red fluorescent protein (RFP)-expressing E. coli and GFP-expressing E. coli together to D. discoideum, and followed their uptake by amoebae over several hours by time-lapsed fluorescence imaging. Amoebae took up and digested uncoated bacteria, while some discoidin-coated bacteria remained intact and were scattered throughout the cytoplasm. The reciprocal experiment with discoidin-coated GFP-expressing E. coli resulted in retention of only those bacteria. The differential handling within the same amoeba of discoidin-coated bacteria versus uncoated bacteria indicated that discoidin must be bound to the bacterium for it to persist after internalization. We quantified the persistence of bacteria after LIMBI by measuring the length of time GFP-labeled bacteria remained intact within vegetative amoebae, using time-lapse imaging in an under agar assay to create a pseudo-two-dimensional view (FIG. 4B)(21). By following bacteria from the time they were internalized by amoebae to the time that they lost their structural integrity, we found that discoidin-coated bacteria persisted nearly 7-times longer than uncoated bacteria (FIG. 4C).

LIMBI may provide an opportunity for bacterial DNA transfer into the host nucleus and we examined this by inducing LIMBI of DscA-coated E. coli harboring a shuttle plasmid and selecting for drug-resistant amoebae. Each of the discoidin-coated E. coli strains introduced into D. discoideum by LIMBI led to genetic transformation of the amoebae at high frequencies (˜2-20%), including those carrying plasmids with G418 [neo^{r}] or blasticidin [bs^{r}] resistance genes (Table 5).

TABLE 5
LIMBI-mediated gene transfer.

| LIMBI conditions¹ | | | RFP-H2b |
|---|---|---|---|
| [plasmid] (treatment) | transformation frequency² | | expression |
| selection | bs^{r} | neo^{r} | (%)⁴ |
| D. discoideum strain AX4 | | | |
| [p53E02] (mock) bs | <0.001³ | — | — |
| [p53E02] (+DscA) bs | 0.17 ± 0.03 | — | — |
| [p14D12] (mock) bs | <0.001³ | — | — |
| [p14D12] (+DscA) bs | 0.18 ± 0.04 | — | — |
| [pRFP-H2b] (mock) neo | — | <0.001³ | <1⁵ |
| [pRFP-H2b] (+DscA) neo | — | 0.026 ± 0.007 | 100⁶ |
| [pRFP-H2b] (mock) none | — | — | <1⁵ |
| [pRFP-H2b] (+DscA) none | — | — | 10 ± 4.6 |
| RAW264.7 murine | | | |
| macrophages | | | |
| [pRFP-H2b] (mock) neo | — | <0.001³ | |
| [pRFP-H2b] (+DscA) neo | — | 0.020 ± 0.0037 | 100⁶ |
| [pRFP-H2b] (+ConA) neo | — | 0.014 ± 0.0026 | 100⁶ |
| [pRFP-H2b] (+SBA) neo | — | 0.018 ± 0.0036 | 100⁶ |

¹E. coli strains harboring the indicated plasmid (pDM304 mCherry-H2b for pRFP-H2b in D. discoideum strain AX4 and mCherry-H2B-C-10 for pRFP-H2b for RAW264 macrophages) were incubated in buffer (mock), or 500 μg/ml DscA lectin, for 60 minutes prior to mixing with for 1 day, followed by selection with blasticidin S (bs, 4 μg/ml) or G418 (neo, 10 μg/ml, or 400 μg/ml for RAW264.7 cells).
²The number of drug resistant D. discoideum colonies observed after 7 days of selection for experiments performed 3 times or more (mean ± s.e.m.).
³No transformants observed in any of 4 selection plates (1,000 cells/plate).
⁴The percentage of cells expressing red fluorescence in their nuclei, indicating expression of histone RFP-H2b as shown in Fig S4B, C) 1 day after LIMBI.
⁵No cells expressed red fluorescence in any field (~100 cells/field) in four experiments.
⁶All cells observed to express red fluorescence (~100 cells/field) in four experiments.

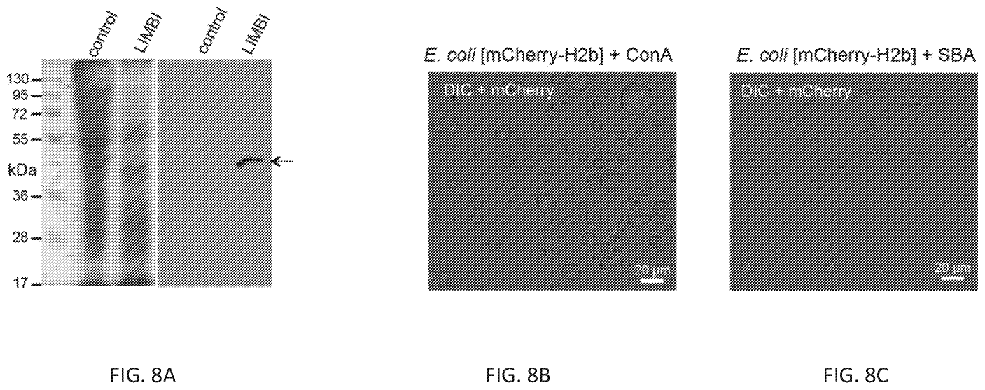

To visualize LIMBI transformation, we used plasmids that express D. discoideum histone H2b fused to the RFP mCherry and observed that all of the transformed amoebae expressed the mCherry-tagged histone within their nulcei (FIG. 4D, Table 5). Immuno-blots of proteins from these cells confirmed that the fusion protein was expressed (FIG. 8A). Somewhat unexpectedly, ˜10% of the amoebae expressed RFP-H2b 24 hours after LIMBI without selection for drug resistance, suggesting that transient expression also occurs (Table 5). These results indicate that DNA can be transferred from the bacteria into the nucleus of the amoebae, suggesting that LIMBI is functionally distinct from the phago-lysosomal digestion pathway that amoebae use to process bacteria as food.

We tested the generality of LIMBI in eukaryotes by testing plant lectins as elicitors and mammalian cells as recipients. We identified several plant-derived lectins that bound to E. coli, including Wisteria floribunda agglutinin (WFA), Dolichos biflorus agglutinin (DBA), Soybean agglutinin (SBA), and concanavalin A (ConA) (22). We mixed lectin-coated bacteria with D. discoideum, using the same protocols developed with discoidin I, and found that each of these plant lectins appeared to be equally effective for LIMBI and LIMBI mediated transformation (FIG. 8B, C; Table 6).

TABLE 6
Conditions for LIMBI

| | | | | Trans- |
|---|---|---|---|---|
| Cells | E. coli¹ | Lectin² | LIMBI³ | formation⁴ |
| Ax4 D. discoideum | [H2b-RFP] | none | N.D. | N.D. |
| Ax4 D. discoideum | [H2b-RFP] | DscA | + | + |
| Ax4 D. discoideum | [H2b-RFP] | WFA | + | + |
| Ax4 D. discoideum | [H2b-RFP] | DBA | + | + |
| Ax4 D. discoideum | [H2b-RFP] | SBA | + | + |
| Ax4 D. discoideum | [H2b-RFP] | ConA | + | + |
| RAW264.7 murine | [H2b-RFP] | none | N.D. | N.D. |
| macrophages | | | | |
| RAW264.7 murine | [H2b-RFP] | DscA | + | + |
| macrophages | | | | |
| RAW264.7 murine | [H2b-RFP] | SBA | + | + |
| macrophages | | | | |
| RAW264.7 murine | [H2b-RFP] | ConA | + | + |
| macrophages | | | | |

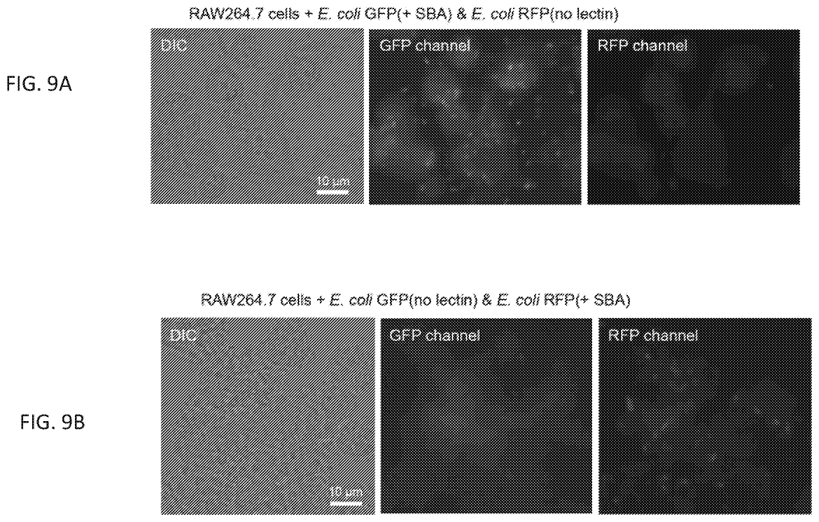

¹E. coli harboring plasmids containing a neomycin resistance cassette and an H2b-RFP expression cassette with Dictyostelium or mammalian transcriptional control elements, as appropriate, were incubated with the indicated lectin prior to LIMBI.
²WFA, Wisteria floribunda agglutinin; DBA Dolichos biflorus agglutinin; SBA, soybean agglutinin; ConA, concanavalin A.
³LIMBI was judged as having occurred (+) when intact bacteria were observed within cells by phase contrast and fluorescence microscopy and ~10% of cells expressed RFP after one day of incubation and prior to selection, as shown in FIG. 9. N.D., not detected.
⁴Transformation was judged to have occurred (+) if drug resistant colonies arose after 1-2 weeks of neomycin (G418) selection and cells expressed H2b-RFP in their nuclei. N.D., not detected.

We also introduced lectin-coated E. coli to mammalian cells in culture and found that DscA, ConA, and SBA were each equally effective for LIMBI and LIMBI transformation of mouse RAW264 macrophages (Tables 5 and 6). Competition experiments with admixtures of GFP- and RFP-expressing bacteria showed that only lectin-coated bacteria persisted intact within RAW264 macrophages after overnight incubation (FIG. 9). Our results show that LIMBI can occur in amoebae and in animal cells, using amoebal or plants lectins, suggesting that modified uptake and persistence of lectin-coated bacteria in eukaryotic cells is a general phenomenon.

We have described physiological differences between D. discoideum carrier and non-carrier strains that appear to account for the salient features of bacterial carriage during social amoebae development. Because discoidin I is sufficient to induce carriage by non-carrier strains, its secretion by carrier strains provides a plausible mechanism for the transition of carrier amoebae from active bacterial killing and feeding, to starvation and bacterial symbiosis. Prestarvation Factor (PSF) is a ˜70-kDa secreted protein that reports on the bacterial food supply at the growth to development transition(23, 24). PSF is sequestered by bacteria when bacterium-amoeba ratios are high, but as the bacteria are depleted by amoebal feeding the level of free PSF rises and stimulates D. discoideum development through autocrine signaling. PSF has never been identified and since discoidin I binds bacteria and affects the kill versus carriage dynamics of amoebae, we are testing whether discoidin I is PSF. Discoidin I's H-type lectin domains are on the C-termini of the trimer subunits, but the N-terminal discoidin domains have distinctive structural similarities to F-type lectins and have been proposed to bind fucose and sialic acid containing N- or O-glycans (14). If true, it is possible that the H-type lectin domains of discoidin I bind bacteria, leaving the discoidin domains free to interact with N- or O-linked glycans on the cell surface of D. discoideum amoebae and initiate LIMBI or engage in receptor mediated signaling. If the uptake mechanism does involve amoebal glycoproteins as receptors, plant lectins might act as bacteria-bound ligands for those same receptors.

Lectinophagocytosis results in efficient bacterial uptake by eukaryotic cells through specific receptor recognition of lectins provided by the bacteria or by the host cell (25, 26). Plant lectins can also mediate macrophage lectinophagocytosis (27, 28) and in some cases this is concomitant with the suppression of bacterial killing (29). It is well known that lectins target bacteria for destruction by the innate immune system of the host, but there are reports of lectins with functions unrelated to defense (30, 31). If lectin protection of bacteria within hosts proves to be a general feature of multicellular eukaryotes, it would provide a new perspective on the regulation of microbiomes in well-studied systems, including humans. For example, LIMBI may promote the maintenance of intracellular bacteria in the mucosal epithelium or myofibroblasts of the colon and contribute to inflammatory disease processes.

### Example 2

### Examples of Materials and Methods for Example 1

Growth and development. Klebsiella pneumoniae (K.p.) was maintained in liquid SM media (32). All laboratory strains of Dictyostelium discoideum were derived from the laboratory strain AX4 (13, 33). D. discoideum wild isolates were from Benabentos et al. (34) or Ostrowski et al. (35). D. discoideum stocks were maintained on K.p. bacterial lawns in co-culture on SM agar (2% Bacto agar (BD Difco) plates made in SM media). AX4 derivatives were also grown axenically in HL5 liquid media supplemented with 50 U/ml penicillin and 50 μg/ml streptomycin (Penn/Strep)(36). Neomycin-resistant strains (neo^{r}) and all of their derivatives were grown in HL5 liquid media supplemented with 20 μg/ml Geneticin® (ThermoFisher). Development of D. discoideum strains was allowed to proceed in situ after growth on SM agar in association with K.p. bacteria. Alternatively, growing amoebea were washed, suspended in KK2 buffer (2.2 g KH₂PO₄, 0.7 g K₂HPO₄ per liter, pH 6.4), plated on 1.5% KK2 agar plates, and incubated for 48 hours to produce fruiting bodies.

DNA constructs. To visualize LIMBI transformation in D. discoideum we constructed a protein-fusion plasmid of D. discoideum histone 2B (H2B) fused to the C-terminus of the red fluorescent protein mCherry, under the control of the D. discoideum actin15 promoter (pDM304 mCherry-H2b). The mCherry gene was amplifed by polymerase chain reaction (PCR) with following primer set.

mCherry 0F_BamHI:
(SEQ ID NO: 1)
5′-GGGGGATCCAAAAAATGGATGGTACTATGGTTAGCAAGGGC
mCherry R_BglIISpeI:
(SEQ ID NO: 2)
5′-CCCACTAGTTTAAGATCTCTTGTACAGCTCGTCCA

mCherry 0F_BamHI was designed with mutations to optimize for D. discoideum codon usage. The resulting PCR amplicon was digested with BamHI and SpeI endonucleases and inserted between the BglII and SpeI restriction sites of pDM304(37) to produce plasmid “pDM304 mCherry”. The H2Bv3 gene of D. discoideum was PCR amplified from the AX4 genome with the following primer set.

H2Bv3-F_BamHI:
(SEQ ID NO: 3)
5′-AAAAGGATCCATGGTATTCGTTAAAGGTCA
H2Bv3-R_SpeI:
(SEQ ID NO: 4)
5′-TTTTTACTAGTTTAGTTTTTGCTTTCAGTTGGATTG

The resulting PCR amplicon was digested with BamHI and SpeI endonucleases and inserted into BglII-SpeI digested pDM304 mCherry to make “pDM304 mCherry-H2b”. The construction was confirmed by DNA sequencing. For LIMBI transformation of mammalian cells we used the mCherry-H2B-C-10 expression plasmid as distributed by addgene (addgene.org, Cambridge Mass.).

Assessment of bacterial carriage during development. We explored the determinants of the ability of social amoebae to retain live bacteria during their multicellular development using assays for the presence of bacteria in the sori of fruiting bodies and by direct observation of live bacteria at various stages of development as described previously to define the original farmer D. discoideum wild isolates(5). We refer to the presence of live bacteria in migrating slugs, in fruiting body sori, and within the spores of D. discoideum using the mechanistically neutral term “bacterial carriage” and the developing amoebal population containing live bacteria as “carriers”. All farmer strains are carriers, by definition, but since it is difficult to determine whether particular laboratory conditions that result in bacterial carriage represent ‘farming’ as described by Brock et al., (5), we separate the fact of bacterial carriage from its potential biological functions for the purposes of this work. We scored fruiting body sori (24-36), individually, for the presence of viable bacteria by picking them with a sterile loop and streaking them on SM agar growth plates, followed by overnight incubation at 37° C. Alternatively, the entire population of sori that form after growth and development on an SM agar plate were transferred by inverting the sori-containing plate over a bacterial growth plate and rapping the pair of plates down onto a few paper towels with enough force so that most of the sori detach from their fruiting body stalks and deposit on the opposing plate as discrete droplets that quickly dry into the plate (FIG. 5B). These plates were also incubated overnight at 37° C. Since Dictyostelium amoebae do not survive at 37° C., the amoebae that might germinate from spores, or amoebae that had not undergone terminal differentiation into spore or stalk, are unlikely to consume bacteria under these conditions. Thus, the appearance of bacterial growth indicates the presence of at least one viable bacterium in the sorus being sampled, and the absence of bacteria suggests that the sorus was devoid of viable bacteria. The total number of sori sampled was ascertained by counting the distinctive small circle of material deposited on the plate after the sorus liquid dried into the plate. Percent carriage is the percent of multicellular organisms that carried bacteria through the developmental cycle, defined as the number of dried sori with at least one bacterial colony divided by the total number of dried sori deposited on the plate. Determinations of bacterial carriage were made in triplicate and the phenotypes and phenomena tested were stable over several years and consistent between multiple investigators.

Biochemical/protein methods. Protein gels and western blots. SDS polyacrylamide gel electrophoresis (SDS-PAGE) was carried out on 12% gels, made from 30% acrylamide/bis-acrylamide (29:1) solution (BioRad). Western blots of SDS-PAGE gels onto nylon filters were carried out with methanol/CAPS transfer buffer (10 mM CAPS pH 10.5, 10% (v:v) methanol) (38). Filters were blocked with Tris-buffered saline (TBS, pH 7.6), 3% BSA, 0.5% Tween-20, for 2 hours at room temperature, and then incubated for 2 hours at room temperature or overnight at 4° C. with monoclonal anti-discoidin IA antibodies (1:10,000 dilution) (39). Mouse monoclonal antibodies to mCherry were obtained from Sigma-Aldrich (#SAB2702291). Filters were washed in TBS without BSA with several changes of buffer over 30 minutes, and incubated for 1 hour with secondary horse radish peroxidase conjugated goat anti-mouse antibodies, washed the filters again and we visualized the signal with a chemiluminescent substrate kit (SuperSignal West Pico #34077, Thermo Scientific).

Purification of Dictyostelium antibacterial proteins (Dab's). Amoebae from 500-ml HL5 cultures (AX4, 10⁶ cells/ml), or scraped from 10 SM plates after growth on lawns of K.p. (wild isolates), were collected by low-speed centrifugation, washed once in Sorenson's buffer (Sor, made from 100× stock solution of 50 gm/L KH₂PO₄, 7.2 gm/L Na₂HPO₄, pH6.0)(40) and the cell pellet was brought up to 40 ml in Sor buffer. K.p. bacteria (10 ml, overnight culture in SM) were collected by centrifugation at 4,000 RPM in a 50-ml conical tube, and the amoebae were added to the K.p. pellet. The amoebae and bacteria were incubated for 2 hours with gentle mixing, after which crude secretomes were prepared by a series of three centrifugation steps. The first was at 1,500 RPM for 5 minutes in a TX-1000 swinging bucket rotor (Sorvall Legend—RT) in order to pellet the amoebae without breaking them. The supernatant was collected by carefully avoiding the pellet of amoebae, and was centrifuged at 4,000 RPM for 5 minutes to pellet the bacteria. The supernatant was again collected by carefully avoiding the pellet of bacteria and centrifuged at 20,000 RPM (ss34 rotor in a Sorvall 4B centrifuge) for 10 minutes at 4° C. The supernatant was then passed through a 0.22-μm pore PES filter (Corning 431097) and the clarified filtrate (10 ml) was then loaded onto a 10-ml DEAE sepharose CL-6B (GE Healthcare) column, washed with 3 column volumes of 10 mM Tris-HCl (pH8) buffer and then eluted with 5, 12-ml steps of 100-, 200-, 300-, 400- and 500-mM NaCl in 10 mM Tris-HCl, pH8, collecting 1-ml fractions. A yield of >90% of Dab activity was generally obtained, always in the 400- and 500-mM NaCl eluate fractions.

Antibacterial activity assays. We used an end-point dilution assay to quantify the antibacterial activity released from amoebae. Overnight cultures of K.p. bacteria grown in SM media were diluted 100,000-fold in SM media, and 50 μl of this suspension was put into wells of a 96-well tissue culture plate (˜1000 K.p. bacteria per well). Samples to be tested were added in a twofold dilution series (dilution into SM media) bringing each well to 100 μl total volume and the plates were incubated at 37° C. overnight and then visually assessed for antibacterial activity. As illustrated in FIG. 6A, turbid growth indicated that the test sample had not killed all of the seed K.p. bacteria, whereas clear wells indicated that all of the bacteria in the well were either killed or were arrested for growth. We tested the viability of the bacteria after incubation by plating the well contents onto SM plates and incubating at 37° C. overnight and confirmed that the bacteria were inviable and not simply arrested in their growth. Antibacterial activity units were defined as the inverse of the last dilution to result in a clear well lacking any observable bacterial growth. The specific activity of a sample was calculated by dividing activity by the amount of protein in the sample, as determined with the BioRad Reagent (41). Specifically, for a 50-μl sample, containing 10 μg of protein, with the 64-fold dilution determined to be the last well with no bacterial growth, the sample would have a specific activity of 6,400 units/mg. To test protection of the bacteria from antibacterials, discoidin I protein was added to K.p. bacteria before adding the antibacterial test samples. We also assessed bacterial killing using short time-course assays where live and dead bacteria were quantified visually. Overnight cultures of K.p. were diluted to an optical density of 1.0 at 600 nm (OD₆₀₀=1). The K.p. suspensions (1 ml) were pelleted by centrifugation at 5,000×g and washed three times with 1 ml sor buffer. The bacterial pellets were resuspended to 60 μl with 1× Sor buffer and containing either 20 μg bovine serum albumin (BSA) as a control, or 20 μg DscA protein. After 1 hour of incubation at room temperature, samples were washed twice in 1 ml Sor buffer, and resuspended in 60 μL of samples containing the antibacterial activity partially purified from D. discoideum secretomes. Aliquots (10 μl) were sampled every 20 minutes and diluted 1:100 into Sor buffer with Baclight LIVE/DEAD stain (Invitrogen) and incubated for 15 minutes. A portion of the suspension (5 μl) was placed in a Petroff-Hauser bacterial counting chamber and live and dead bacteria were quantified by fluorescence microscopy. Differences in viability between the samples were tested for statistical significance at each time point using a one-way pairwise ANOVA using Tukey contrasts for multiple comparisons of means within the R Project for Statistical Computing and the R-commander package (42).

Discoidin I protein purification. Mixtures of discoidin IA and discoidin IC (DscA/C) were purified from the secretomes of D. discoideum strains, and pure discoidin IA (DscA) was purified after heterologous expression in E. coli as a hexahistidine N-terminal fusion protein (see “DNA constructs” above). Secretomes from D. discoideum prepared as described above, or clarified supernatants from E. coli induced to over-express DscA, were subjected to successive rounds of poly-galactose affinity chromatography on Sepharose CL-4B columns, as described previously (43). The purity of the preparation is illustrated in FIG. 3A (left lane, “1× S”) and we assume that the final fractions contain a high percentage of properly folded proteins since the purification relies on the lectin binding function of discoidin I. Discoidin was stored in working stock solutions of 1 mg/ml in Sor buffer.

Discoidin I binding to bacteria. Lectin binding experiments were carried out in 100 μl Sor buffer containing 1.33×10⁸ K.p., or E. coli, bacteria and 0-60 μg of purified DscA. The number of bacteria was obtained by counting in a Petroff-Hausser chamber and confirming that number by determining the number of colony forming units of serial dilutions of the overnight cultures used in the assays. The pellet of bacteria was resuspended in Sor buffer and variable amounts of DscA (1 mg/ml in Sor buffer) was added to bring the volume up to 100 μl, and incubated with gentle shaking for 1 hour at room temperature. The bacteria were pelleted by brief sedimentation in a microfuge, and the unbound DscA was measured using the BioRad Reagent (41). Bound DscA was inferred by subtracting the unbound DscA from the total DscA added to the reaction. Reactions were carried out in triplicate and on several batches of DscA, each with similar results.

We used the method of Steck and Wallach to estimate binding parameters (44), as described by Reitherman, et al. for discoidin proteins binding to D. discoideum amoebae (20). This produced an apparent association constant of 1.2×10⁶ M⁻¹, and 1.4×10⁶ DscA protein trimers bound per bacterium. As a check of the Steck/Wallach model we used a simple way to estimate the number of DscA proteins bound by taking the y-intercept of the asymptote from the binding curve in FIG. 3B as the maximum DscA binding. This is 20 μg of DscA in this assay, which equates to 0.15 pg/bacterium, or 0.9×10⁶ DscA trimers per bacterium, so the two methods agree fairly well.

We also estimated the number of DscA trimers needed to coat the K. pneumoniae surface. The volume of a protein is given by MW (Da)/825=V(45). Using 84,774 Daltons as the molecular weight of a DscA trimer, its volume is 102.75 nm³. Assuming that the roughly cylindrical trimer structure is twice as long (L) as it is wide (2r) (14), or L=4r, the area of the lectin binding end is ˜12.74 nm² [V=πr²L, or V=4πr³, so 102.75 nm³=4πr³, and r=2.01465 nm. Therefore, A=πr²=π(2.01465)², or 12.74 nm²]. The dimensions of the Gram(−) rod bacterium K. pneumoniae is L=2 μm, r=0.25 μm, so the surface area would be A=2πrL+2πr², or 3.53 μm² (3.53×10⁶ nm²). Thus, we can estimate that ˜277,000 discoidin I trimers could occupy the surface of K. pneumoniae assuming a smooth surface and end-on binding geometry of discoidin I (14). This simplified calculation provides a rough estimate of the number of DscA trimers needed coat the surface of K. pneumoniae, as if the trimers were microvilli protruding at right angles from an idealized cell surface.

Sentinel cell quantification. Growing cells were harvested SM agar, K.p. growth plates by low-speed centrifugation and resuspended in Sor buffer. Cells were plated for synchronous development and slug migration on agar petri plates (6-cm) made with 12 ml Sor-buffered 1% washed agar (Noble Agar, BD Difco) and with 3 μg/ml ethidium bromide added just prior to pouring the plates (EB-agar). For some experiments EB concentrations were varied from 0.1-100 μg/ml. 50 μl of cell suspension (10⁹ cells/ml) was deposited in a line on one side of each plate. Plates were placed in a dark humid chamber (22° C.) with a unidirectional light source where slugs were allowed to form and migrate for 24-48 hours. Slugs from several plates were collected into 1 ml of Sor buffer, after they had migrated away from the position of the original cell deposition. Slugs cells were dispersed by trituration using a Gilson micropipetor fitted with a standard 1000-μl disposable pipet tip, collected by centrifugation and suspended PDF buffer (22.2 mM potassium phosphate, 20 mM KCl, 1.0 mM CaCl₂, 2.5 mM MgSO₄, pH 6.4(36)) containing 2% sucrose, an equal volume of the fixation solution was added (4% paraformaldehyde, 30% Picric Acid, 10 mM PIPES, pH 6.5), followed by gently mixing for 15 minutes. After fixation cells harvested and resuspended in 0.2 ml 2% sucrose in PDF and stored at 4° C. for later flow cytometry. Flow cytometry was carried out as described previously, in the Baylor College of Medicine Advanced Technology Core laboratory for cell sorting, using a Beckman-Coulter Altra cell sorter, adjusting the parameters for the measurement of single cells using the forward scatter plot as a guide (3, 46). S cells display greater than 30-fold higher average fluorescence compared to other slug cells so we set the window used to count S cells that largely avoided inclusion of non-S cells and included a population that was >95% S cells. To validate the window parameters, S cells were collected from this window and confirmed to be >95% S cells by direct microscopic visualization. We sorted cells into 2% sucrose in PDF buffer and fixed them with an equal volume of 4% formaldehyde. The cells were spun onto microscope slides using a Cytopro 7620 cytocentrifuge (Wescor) and mounted with DAPI containing Vectashield (H-1000, Vector labs, Inc.). Preparations were visualized on a Delta Vision deconvolution microscope and images were processed with the SoftWoRx (version 2.5) software package (Applied Precision, Issaquah, Wash.).

Lectin-induced modified bacterial internalization (LIMBI). To assess LIMBI mediated bacterial carriage during D. discoideum development, amoebae (5×10⁵ in 0.5 ml HL-5) were mixed with lectin-coated K.p. (0.1 ml) and plated on 10-cm SM agar culture plates. Bacteria were prepared from overnight cultures of K.p., adjusted to OD₆₀₀=2, and 600 μl was pelleted to remove most of the growth media. Lectin protein (30 μg), or BSA as a control, was added to the ˜60-μl bacterial pellet in 100 μl final volume and incubated for 1 hour at room temperature with slow shaking. After lectin binding, D. discoideum amoebae were added directly into the bacterial suspension before plating for development as described above. The lectins used were a mixture of discoidin A and C, purified from D. discoideum, a hexahistidine N-terminal fusion of discoidin IA (DscA) purified from E. coli, and plant lectins, obtained from Sigma-Aldrich, from Wisteria floribunda agglutinin (WFA, L8258), Dolichos biflorus agglutinin (DBA, L2785), Soybean Agglutinin (SBA, L1395), concanavalin A (ConA, L7647). Under these conditions fruiting bodies were observed after 36 hours, but bacterial carriage was tested by harvesting fruiting body sori after 48 hours using the bacterial carriage assays described above. To image bacteria inside spores, we spread sori onto slides and fixed them with formaldehyde (4%) in methanol, and captured images with a Leica confocal microscope system (TCS-SP5) using a 100× objective. LIMBI was visualized in vegetative cells in an under agar assay in order to minimize spatial complexity in the z-axis and for better image resolution (21). We mixed lectin-coated (or mock treated) GFP-expres sing E. coli with amoebae at an approximate 5:1 ratio and plated the mixtures onto 60-mm KK2-buffered agar plates, allowing the suspension to dry into the plate for <1 hour. Blocks with the LIMBI cell mixture were cut from the agar plate and placed upside-down on glass-bottomed dishes (MatTek corporation). Time-lapse images were acquired every minute for 3 hours under fluorescence and DIC microscopy on a Nikon inverted microscope system (Eclipse Ti) using a 100× objective. Assessment of the persistence of the bacteria after uptake by amoebae was accomplished manually. Under agar almost all of the bacteria are found in the spaces surrounding the amoebae and not between the glass and the amoebae or between the agar and the amoebae. This was confirmed by manual inspection along the z-axis. Bacteria were followed from the time of uptake by phagocytosis until they lost their structure integrity, revealed by their transition from green puncta to faint clouds of GFP signal. The transition occurs presumably because the bacteria have been killed and the GFP is dispersed as the bacterial cell is compromised on its way to being degraded. Dozens of cells were tracked in this way in several experiments and lectin-coated bacteria were consistently observed to have longer persistence times as described in the results.

LIMBI Transformation. Transformation of D. discoideum. Two different protocols were developed that gave equivalent results. In the first, LIMBI is followed by incubation on SM nutrient agar plates, with a short period of co-culture of amoebae and bacteria. In the second protocol, LIMBI is followed by incubation on buffered agar, allowing enough time for the bacteria to be cleared from the plate. Though we believe LIMBI occurs immediately, there is also concomitant digestion of bacteria under each of these conditions. Significant transient transformation can occur prior to applying selection, as it is possible to detect expression in D. discoideum from the E. coli plasmid 24 hours after the start of the procedure (Table 5). We have not yet carried out systematic analyses of LIMBI transformation efficiency using different strains of E. coli or other E. coli-D. discoideum shuttle vectors. Discoidin I binding to bacteria occurs with low affinity (˜10⁶ M⁻¹ for K.p.), so the initial binding is carried out in a small volume at a near-saturating concentration of discoidin I in order to drive the lectin onto the bacteria (>0.3 mg/ml discoidin I). We have used a hexahistidine N-terminal fusion of discoidin IA (6× His-DscA) produced in E. coli (see above), as well as a mixture of discoidin IA and IC (DscA/C) purified from the conditioned buffer obtained from wild carrier strains after they had been scraped from SM growth plates at the time of the growth-development transition and shaken in buffer for 3 hours. We have detected no differences between 6× His-DscA and DscA purified from amoebae in any of our experiments. We used AX4 amoebae for LIMBI transformation harvested during exponential growth in HL5 (36), and we used E. coli strains harboring the plasmids described in Results.

LIMBI transformation on SM agar plates. For each transformation pellet the bacteria in 0.6 ml of an overnight culture (adjusted to OD₆₀₀=1.5-2.0) by centrifugation at 12,000 rpm, for 5 minutes in a microfuge and discard the supernatant. Add purified discoidin I (30 μl, 1 mg/ml in sterile Sor buffer) or plant lectin (SBA, WFA, DBA, ConA) to resuspend the bacterial pellet and bring the volume up to 100 μl with Sor buffer. We mock treated samples with buffer, for use as negative controls. We incubated the bacteria-lectin mixture at room temperature for 60 minutes with shaking. We mixed 0.5×10⁶ exponential phase AX4 cells, in 0.5 ml SM media, with the lectin-coated bacteria and spread the mixture evenly onto one 100-mm SM plate (or onto one 60-mm KK2-buffered agar plate, see alternate protocol below). We incubated the plates at room temperature for ˜1.5-2 days until the bacterial lawn was cleared and then collected the amoebae, avoiding as much of the residual bacterial lawn as possible, and resuspended them in 10 mls of HL5. We washed the cells once with HL5 and resuspended them in 1 ml HL5. To determine the LIMBI transformation efficiency, we diluted amoebae (100,000, 10,000, 1,000, 100) into 10 ml of HL5 with antibiotics [10 μl chloramphenicol (25 mg/ml); and either 10 μl carbenicillin (50 mg/ml) for kanr plasmids, or 10 μl kanamycin (50 mg/ml) for ampr plasmids] and plated them into a standard plastic tissue culture plate. After 16-20 hours, we added the transformation selection drug; 10 μl blasticidin (4 mg/ml) for bs^{r} plasmids, or 5 μl G418 (20 mg/ml) for neon plasmids. Colonies of transformed amoebae were observed after 7-10 days of incubation at room temperature. Since LIMBI transformation occurs at high efficiency, we observed distinct colonies only when we plated fewer than 5,000 amoebae.

LIMBI transformation on KK2-buffered agar plates. This protocol is the same as the SM agar protocol except that after the lectin-coated bacteria are mixed with the AX4 amoebae, the mixture and spread evenly onto one 60-mm KK2-buffered agar plate (pH 6.4). We incubated the plates overnight at room temperature (˜16 hours) until most of the bacteria were internalized and before the amoebae began to aggregate. We collected the amoebae by scraping them from the plate and suspending them in 10 ml of HL5. We then followed the plating and selection procedure described above for SM agar plates. Note that LIMBI with ConA results in some multinucleated amoebae and this persists even after 10 days of culture (FIG. 8B).

LIMBI Transformation of mammalian cells. We carried out LIMBI transformation of RAW264.7 cells (ATCC® TIB71™) with mCherry-H2B (Addgene.org plasmid mCherry-H2B-C-10). We have transformed MA104 monkey kidney epithelial cells (ATCC® CRL-2378.1) with similar results. For each transformation, 10⁶ lectin-coated E. coli were added to 2×10⁶ RAW264.7 cells in wells of a 6-well tissue culture plate (DMEM media supplemented with 10% fetal calf serum, incubated at 37° C. in 5% CO₂). E. coli were coated with lectin as described above for transformation of amoebae. The bacteria-cell mixtures were incubated overnight, the plates were then washed twice with PBS to remove most bacteria, treated with trypsin (0.25% in EDTA) to free the adherent cells, the cells were collected and washed to remove trypsin, and suspended in fresh media. We then aliquoted cells into wells of 96-well plates, with 400 μg/ml G418 and antibiotics [chloramphenicol (25 μg/ml), kanamycin (50 μg/ml)]. To visualize expression of mCherry-H2B cells were loaded onto slides and then fixed with methanol DAPI 1:1000. Image on a Nikon inverted microscope (eclipse Ti) in DIC and fluorescence channels.

### Example 3

### Social Amoebae Establish a Protective Interface with Their Bacterial Associates Bylectin Agglutination

Animals and amoebae share conserved cellular functions for immune defense such as phagocytosis and DNA-based extracellular traps that they use to kill bacteria. Whether they also share tissue-level barriers to protect them from direct exposure to bacteria is not known. We have explored this question in the social amoebae Dictyostelium discoideum that can form plaques on thick lawns of food bacteria that expand as amoebae divide and bacteria are consumed. In this Example, we show that CadA, a cell adhesion protein that functions in D. discoideum multicellular development, is also a bacterial agglutinin that forms a protective interface at the plaque edge to limit the exposure of vegetative amoebae to bacteria. This interface is important for amoebal survival when bacteria-to-amoebae ratios are high, optimizes amoebal feeding behavior and protects amoebae from oxidative stress. Lectins also control bacterial access to the gut epithelium of mammals to limit inflammatory processes, so this appears to be common strategy of antibacterial defense across a broad spectrum of eukaryotic phylogeny.

CadA is Important for Viability when Amoeba Grow on High-Density Bacteria

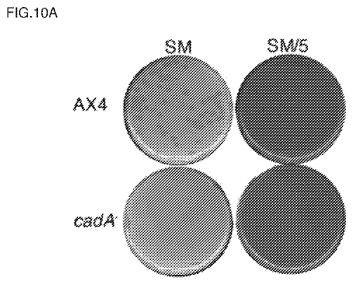

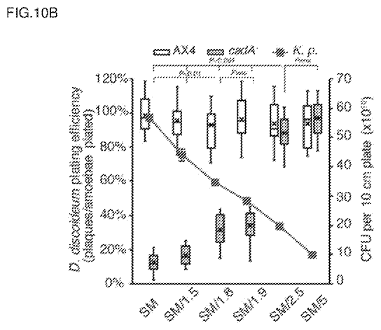

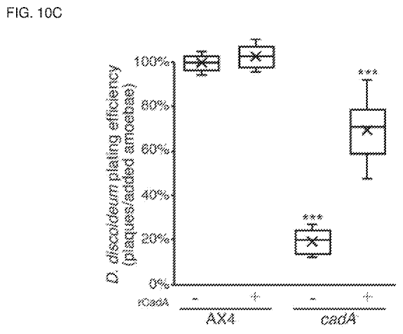

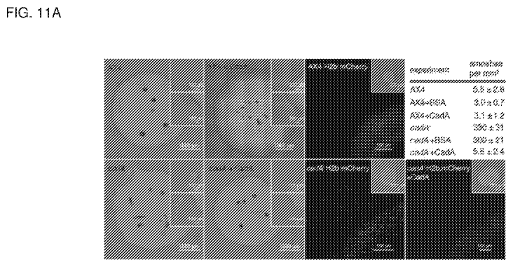

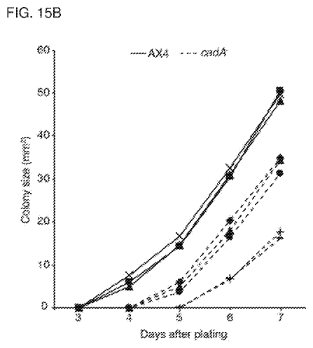

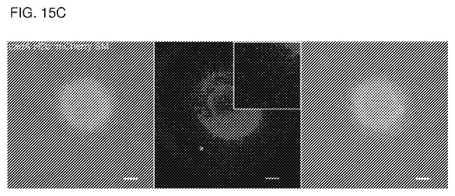

When individual D. discoideum amoebae grow on lawns of food bacteria they form circular plaques as the amoebal colony expands through rounds of cell division (FIG. 10A). When working with cadA− mutant amoebae described previously we noticed that their plaques were variable in size, suggesting they had a growth defect (Wong et al., 2002). To explore this, we deleted the coding sequence of cadA in our laboratory strain AX4 and measured the rate of expansion of cadA mutant plaques from the time of their delayed appearance on the bacterial lawn and found that the rate was indistinguishable, on average, from the laboratory wild-type strain AX4 (FIG. 15A). Since the cadA− mutant amoebae grew normally once the plaque had formed, this suggested that they initiated plaques inefficiently. Testing this directly, we found that <20% of cadA− mutant amoebae form plaques under standard laboratory growth conditions in co-culture with K. pneumoniae on SM agar plates (FIGS. 10A, 10B). However, the viability of the cadA− mutant was indistinguishable from the parental strain when co-cultured with M. luteus, B. subtilis, or S. aureus (FIG. 15A). The low viability of cadA− amoebae when cultured with K. pneumoniae and their normal viability when cultured with Gram(+) bacteria suggested that secreted CadA is important for plaque formation on at least some species of Gram(−) bacteria. A role for CadA during D. discoideum's growth stage was unexpected since it has a well-characterized role as a cell adhesion protein during multicellular development (Knecht et al, 1987; Lin et al., 2006; Sriskanthadevan et al., 2011). Mutants of D. discoideum that are defective in their responses to Gram(−) bacteria display reduced viability on dense lawns of K. pneumoniae bacteria, but grow normally on dead K. pneumoniae (Nasser et al., 2013; Chen et al., 2007). This indicates that growth on a preferred food bacteria like K. pneumoniae can be toxic for amoebae. The low viability of cadA− mutants might also be due to K. pneumoniae toxicity so we compared their viability during co-culture with K. pneumoniae on SM agar plates containing successively lower nutrient levels, which produced bacterial lawns that were successively less dense (FIG. 10B). Indeed, the cadA− amoebae were about twice as efficient in forming plaques on SM/1.8 agar (1.8-fold diluted nutrients; FIG. 10B). On SM/2.5 and SM/5, the plating efficiency of the cadA− amoebae was indistinguishable from the parental AX4 (FIG. 10A, 10B). These data suggest that cadA is necessary for the survival of amoebae when co-cultured with dense bacteria, but it is also possible that K. pneumoniae bacteria are less toxic to amoebae when they are grown under sub-optimal nutrient conditions. To address this, we tested whether CadA protein could rescue the viability of the cadA− amoebae with bacteria growing under standard nutrient conditions. We applied recombinant CadA, purified from CadA-expressing E. coli, to cadA− mutant amoebae and plated the mixture on SM agar plates seeded with K. pneumoniae. Interestingly, cadA− mutant amoebae produced >3-times more plaques when exogenous CadA was present; up to ˜70% of the viability of the wild-type parent (FIG. 10C). Taken together, these results suggest that extracellular CadA is import for the survival of D. discoideum amoebae during the initial stages of plaque formation.

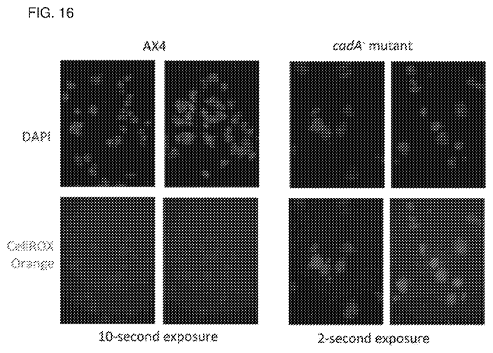

The compromised viability of cadA mutants suggests a role for the CadA protein in protecting the amoebae from bacterial toxicity. To test this, we examined the production of reactive oxygen species (ROS) in amoebae as one indication of toxic stress. We sampled amoebae feeding on bacteria at the plaque edge and measured ROS using a ROS-activated dye that becomes fluorescent orange when exposed to ROS. By this measure, the cadA mutant amoebae had much higher levels of ROS compared to wild-type cells (FIG. 16). This suggests that CadA suppresses the toxicity of K. pneumoniae to D. discoideum amoebae that are actively feeding at the plaque edge.

CadA Prevents Mixing of Bacteria and Amoebae Across Plaque Borders.

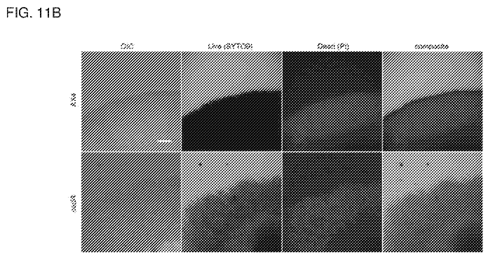

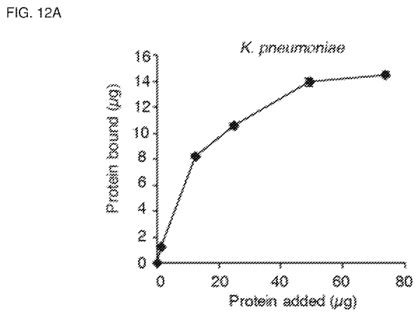

When cadA− amoebae produced plaques on low-density K. pneumoniae lawns they were irregular in shape with many small satellite plaques outside of the borders of the main plaques; they lacked the symmetry and distinct borders of wild-type plaques (FIG. 11A). Satellite plaques are occasionally formed by wild-type, when motile amoebae migrate into the bacterial lawn beyond the edge of the main plaque. To visualize the dispersal of amoebae beyond the plaque border, we labeled their nuclei with a protein fusion of histone H2b and a red fluorescent protein, H2bmCherry. We examined plaques on lawns of K. pneumoniae by fluorescence microscopy and observed many cadA− amoebae beyond the growing edge of every mutant plaque, and very few AX4 amoebae outside of any wild-type plaque (FIG. 11A). We also found hundreds of cadA− amoebae outside of mutant plaques on thick lawns of K. pneumoniae (SM/1.9 and higher), but they must have low viability since very few satellite plaques ever formed though hundreds of cells outside of every plaque but (FIG. 15C). To test if this phenotype resulted from a lack of extracellular CadA, we mixed CadA with cadA− amoebae and spotted the mixture on SM/2.5 agar plates seeded with K. pneumoniae. The cadA− mutant plaques formed in the presence of CadA protein were symmetrical, with very few cells migrating beyond the plaque edge, and they resembled wild-type plaques (FIG. 11A).

Most bacteria found within D. discoideum plaques are dead; presumably killed by antibacterial proteins secreted by the amoebae (Dinh et al., 2018). Since cadA− amoebae do not remain within the borders of plaques we examined whether or not bacteria remained within the bacterial lawn surrounding the plaque. We visualized the bacteria at the growing edge of mutant and wild-type amoebal plaques growing on K. pneumoniae lawns on SM/2.5. While the growing edges within AX4 plaques contained mainly dead bacteria, the edges of cadA− plaques contained patches of live bacteria (FIG. 11B). Note that D. discoideum amoebae appear free of any fluorescence, presumably due to robust transporters pumping out the dyes (Good et al., 2000; Anjard and Loomis, 2002). These results suggest that CadA is necessary and sufficient for proper organization of bacteria and amoebae at the growing edge of D. discoideum plaques, establishing a border between them.

CadA is a Bacterial Agglutinin that Restricts Expansion of Bacterial Colonies.

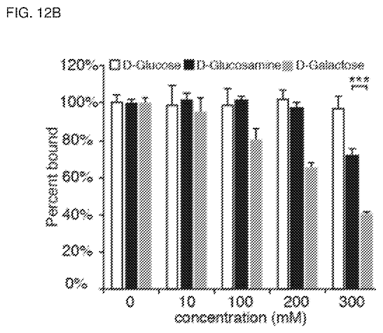

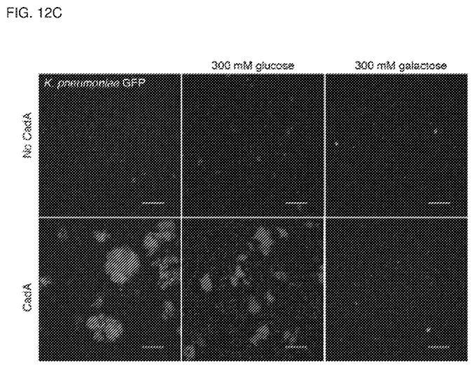

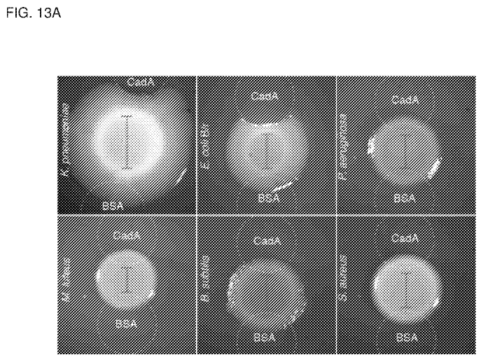

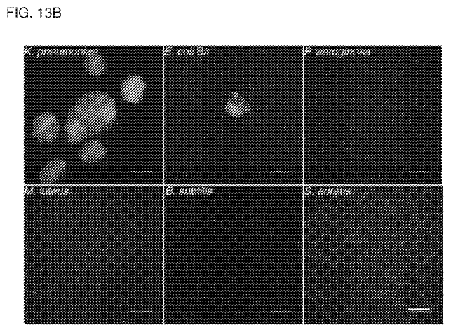

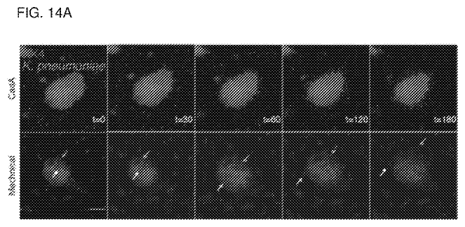

We recently showed that the D. discoideum lectin discoidin I binds to K. pneumoniae and modulates the interactions of bacteria and amoebae (Dinh et al., 2018). Our results suggested that CadA also interacts directly with bacteria so we examined CadA's ability to bind K. pneumoniae. Using recombinant CadA protein and a bacterial binding assay, we observed saturable binding of CadA to K. pneumoniae and could estimate an apparent Ka of 1.0×107 M-1 and 7.5×105 binding sites per bacterium (FIG. 12A). To begin to characterize the CadA binding target on the bacteria, we attempted to inhibit CadA binding with various monosaccharides. We observed significantly reduced CadA binding to K. pneumoniae in the presence of 100 mM Galactose and 300 mM Glucosamine (FIG. 3B). This suggests that CadA is a lectin that binds to carbohydrates on the surface of K. pneumoniae, perhaps to their lipopolysaccharide which is rich in galactose moieties (Vinogradov and Perry, 2001).

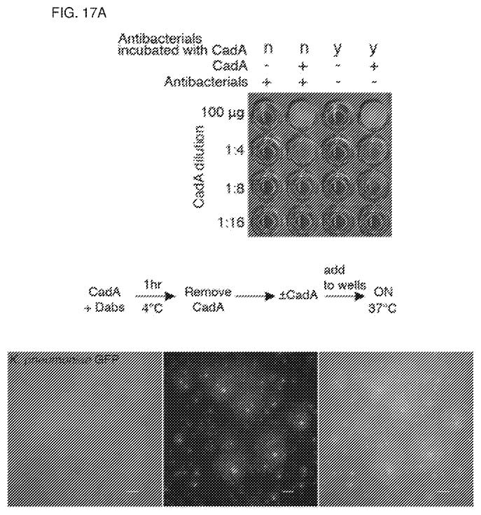

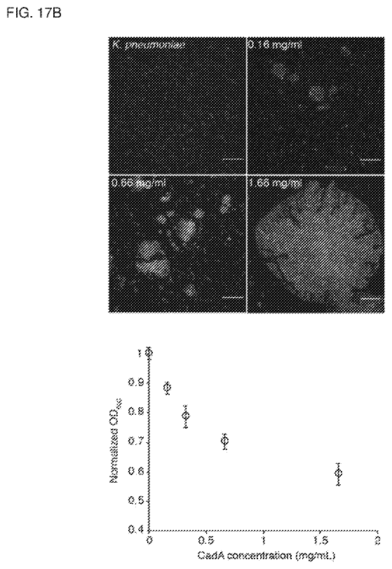

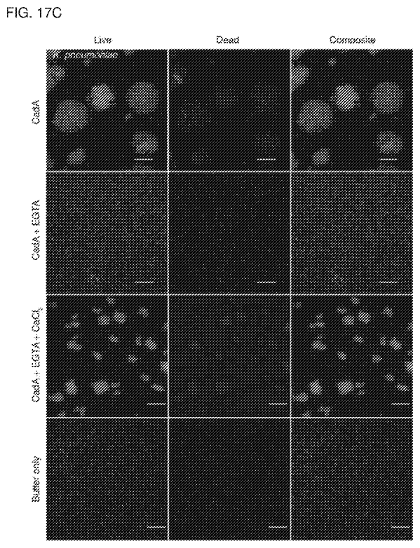

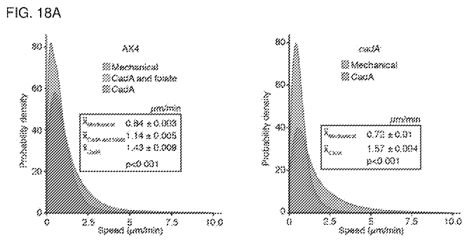

We next tested whether CadA binding protects K. pneumoniae bacteria from D. discoideum antibacterial proteins, as we recently demonstrated for discoidin I (Dinh et al., 2018). We isolated Dictyostelium antibacterial proteins (Dabs) that are naturally secreted by AX4 during feeding on K. pneumoniae bacteria, as described previously (Dinh et al., 2018). We mixed CadA with K. pneumoniae bacteria suspended in growth media containing partially purified Dabs. Without added CadA we observed complete killing of the bacteria, as expected, but at higher CadA concentrations we observed robust bacterial growth suggesting that CadA had protected the bacteria from killing (FIG. 17A). To ensure that CadA had not inactivated or sequestered the Dabs we incubated them together, prior to the assay, and after removing CadA the Dabs retained their ability to kill bacteria, suggesting that CadA protection works through direct interaction with bacteria (FIG. 17A). By microscopic examination of the assay wells we observed non-uniform bacterial growth at CadA concentrations that were protective to the bacteria. Using K. pneumoniae expressing green fluorescent protein (GFP) in the protection assay, we observed a few concentrated foci of surviving bacteria adhering to the surface of the plate (FIG. 17A), suggesting that CadA may protect the bacteria by sequestering them within clumps, possibly reducing their exposure to the Dabs. We directly tested CadA for agglutinating activity by incubating K. pneumoniae suspensions with increasing concentrations of CadA protein and followed bacterial clump formation by light scattering and by direct microscopic examination. We observed bacterial clumps at 0.16 mg/ml CadA and larger clumps were produced as we increased the concentration of CadA (FIG. 12C; FIG. 17B). Structural studies of CadA revealed bound calcium ions in the N-terminal domain that are necessary for amoebal cell-cell adhesion (Lin et al., 2006). To determine whether calcium is necessary for bacterial agglutination, we incubated CadA with the calcium chelator EGTA for 1 h and then removed the EGTA. The agglutinating activity of EGTA-treated CadA was greatly diminished compared to untreated protein, but could be restored by adding calcium chloride to the assay, indicating that calcium is necessary for agglutination (FIG. 17C). Finally, bacterial agglutination was completely abolished by 300 mM galactose suggesting that the lectin function of CadA is necessary for bacterial agglutination (FIG. 12C).

The agglutinating activity of CadA provides a potential mechanism for its role in amoebal survival on dense bacterial lawns. One possibility is that CadA forms a lattice out of the proximal bacteria at the plaque edge, forming a barrier that modulates the exposure of amoebae to bacteria. To explore this idea we attempted to produce a bacterial barrier with pure CadA protein without any amoebae present. We deposited recombinant CadA protein on the surface of nutrient agar plates and spotted bacterial suspensions adjacent to the protein deposits. As the K. pneumoniae, or E. coli B/r, bacteria grew and expanded beyond the initial deposition of cells they were unable to occupy areas containing CadA protein, but they did expand into spots of a control protein, bovine serum albumin (BSA, FIG. 13A). Bacterial spots of the other species that we tested expanded symmetrically into the areas of CadA and BSA protein deposition (FIG. 13A). CadA was able to agglutinate E. coli B/r, as described for K. pneumoniae above, but CadA did not agglutinate S. aureus, B. subtilis, P, aeruginosa, or M. luteus (FIG. 13B). Thus, CadA's ability to restrict colony expansion of a give bacterial species correlated with its ability to agglutinate those bacteria (compare FIGS. 13A and 13B). These results, together with the plaque phenotype of cadA mutants, suggest that Dictyostelium amoebae secrete CadA to form a dynamic interface at the plaque edge to control their interaction with K. pneumoniae bacteria.

Altered Amoebal Predation of CadA-Agglutinated Bacteria.

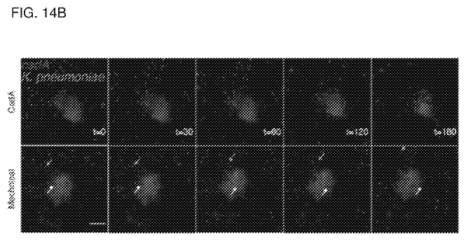

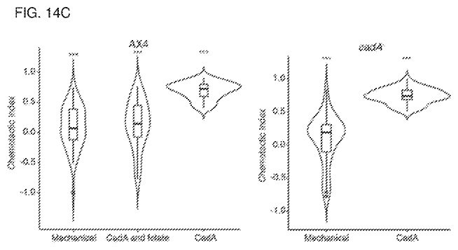

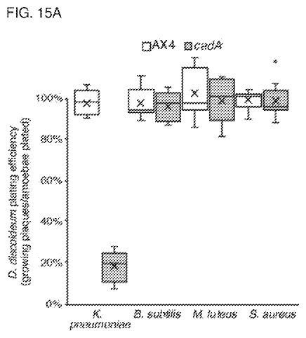

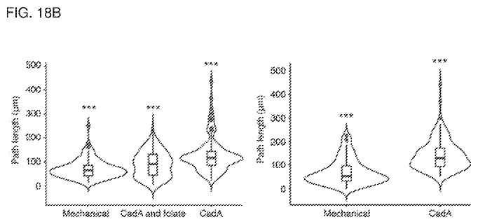

The agglutination of bacteria at the edge of Dictyostelium plaques would be expected to affect amoebal predation and feeding. We examined the feeding behavior of amoebae with a model of the plaque edge that has the inverse geometry of a plaque; amoebae surrounding CadA-agglutinated clumps of bacteria. We observed amoebae presented with a mixture of CadA-agglutinated K. pneumoniae bacteria and planktonic bacteria using an under-agar assay for optimal visualization. As a control we presented amoebae with clumps of bacteria that were generated mechanically from packed pellets formed by centrifugation of bacterial suspensions. Amoebae actively moved toward and surrounded the CadA-induced bacterial clumps and as they divided the daughter cells remained feeding at the edge of the same clump (FIG. 14A, 14B). Amoebae approached mechanically-generated clumps chaotically, some penetrated to the interior of clumps, and some left the clumps altogether (FIG. 14A, 14B). Both wild-type and cadA− mutant amoebae chemotaxed more efficiently toward the CadA-induced clumps as compared to the mechanicallygenerated clumps (FIG. 14C). Directed movement of amoebae toward CadA-induced clumps appears to represent folate chemotaxis since the presence of 100 μM folate abolishes the effect (FIG. 14C). These results suggest that CadA-induced clumping provides agency to social predation of bacteria. To test if CadA-agglutinated clumps of bacteria displayed enhanced attraction to amoebae, we compared the speed the amoebae approaching clumps of K. pneumoniae formed by CadA with clumps formed by mechanical means. Tracking individual cells predating on a single clump of K. pneumoniae, we observed both AX4 and cadA− amoebae move faster toward CadA-induced clumps and over longer distances compared to mechanically-induced clumps (FIG. 18). Interestingly, the addition of 100 μM folate only partially reduced the speed of amoebae approaching CadA-induced clumps FIG. 18A). These data suggest that CadA agglutinates bacteria in a way that signals amoebae to prey on bacteria cooperatively and in a way that should limit their exposure to the harmful effects of live bacteria.

Examples of Methods

Strains, growth, and plasmids. Klebsiella pneumoniae (K.p.) was grown in SM media or on SM agar (Backhed et al., 2005). Dictyostelium discoideum strains were derived from the axenic laboratory strain AX4 (Haag, 2018; Belkaid and Hand, 2014). and maintained in HL5 media with 50 U/mL penicillin and 50 μg/mL streptomycin (Penn/Strep) (Kim et al., 2018) or in co-culture with K.p. on SM agar plates (2% Bacto agar (BD difco) with SM media) (Belkaid and Hand, 2014). Blasticidin-resistant D. discoideum strains (bsr) were grown in HL5 media supplemented with 4 μg/ml Blasticidin (ThermoFisher). Neomycin-resistant strains (neor) were grown in HL5 media supplemented with 20 μg/ml Geneticin® (ThermoFisher). DNA constructs. pDM304 mCherry-H2b was transformed into AX4 and cadA mutant strains in order to fluorescently tag their nuclei (Dinh et al., 2018). Construction of the cadA knockout vector pLPBLP_cadA was accomplished using the following primers and homologous recombination.

5′ homologous arm was amplified with:
cadA_HA5_fwd KpnI/BbsI:
(SEQ ID NO: 5)
aaaggtaccgaagacTTCCTGATGGTGATGATGGTTATGA
cadA_HA5_rev SalI:
(SEQ ID NO: 6)
tttgtcgacATTCAAATGATTCACCAGTGCAGTT
3′ homologous arm was amplified with:
cadA_HA3_fwd BamHI:
(SEQ ID NO: 7)
aaaggatccAGACATTCCCAAAGAATATGACTGT
cadA_HA3_rev SpeI/BbsI:
(SEQ ID NO: 8)
tttactagtgaagacAAAAAAAATTTCCCGCTTTGAAGGG

Homologous arms were amplified from AX4 gDNA. 5′ homology arm and pLPBLP vector were separately digested with KpnI and SalI and ligated together. The resulting vector and the 3′ homology arm were digested separately with BamHI and SpeI and ligated. pLPBLP_cadA was digested with BbsI resulting in linear fragments containing only homologous DNA and the blasticidin resistance cassette. The linear DNA was electroporated into amoebae and mutants were selected with 10 μg/mlblasticidin (Sutoh, 1993).

Plating efficiency and plaque growth. Plating efficiency assay was performed by plating 100 amoebae with bacteria onto nutrient agar plates. After 4 days, the number of observed plaques was counted. Plating efficiency was calculated by dividing the number of observed plaques by the number of plated amoebae. To measure plaque growth, 50 amoebae were added to 400 μl of overnight K. pneumoniae culture and plated on SM agar. Plates were observed until “pin prick” plaques became visible. The diameter of each plaque was then tracked for 4 days and area was calculated (area=π*(d/2)²).

Measurement of reactive oxygen species. Amoebae were mixed with overnight cultures of K. pneumoniae and plated on SM agar. After 4 days of growth, amoebae and K. pneumoniae bacteria were collected from the plaque edges with a sterile loop and placed on Sor agar. CellROX® Orange (Thermofisher) was added to a final concentration of 5 μM and incubated for 30 minutes prior to fluorescence imaging.

Expression of His6-tagged CadA. His6-tagged CadA was expressed from the pETMCadA vector from Dr. Chi-Hung Siu (Lin et al., 2006). Overnight cultures were diluted 1:100 into LB with 50 ug/mL Carbenicillin at 37° C. Cultures were grown to an OD₆₀₀ of 0.8. Protein production was induced with 0.4 mM IPTG for 3 hours. Cells were spun down at 14,000 rcf for 20 mins at 4° C. Pellets were resuspended in sonication buffer containing 1 mg/mL lysozyme. The bacteria were sonicated on ice to lyse. Samples were centrifuged at 20,000 rcf for 20 minutes at 4° C. The supernatant was collected and CadA was purified using HisPur™ Ni-NTA resin (ThermoFisher) and the corresponding protocol. Samples were then concentrated, and buffer exchanged with Sorenson's buffer (2.0 g KH2PO4, 0.29 g Na₂HPO₄ per liter, Sor) using 3,000 kDa molecular weight cutoff spin column (Centricon Ultracel®-3K).

Biochemical methods. SDS polyacrylamide gel electrophoresis (SDS-PAGE) was performed on 12% gels with BioRad 30% acrylamide/bis-acrylamide (29:1) solution. Western blot transfer from SDS/PAGE gels to nitrocellulose membranes were performed with methanol/Tris transfer buffer (25 mM Tris pH 7.6, 20% (v:v) methanol). Membranes were blocked with Tris-buffered saline with Tween-20 (TBST, pH 7.6, 0.5% Tween-20) and 5% nonfat milk for one hour at room temperature. Monoclonal anti-CadA antibodies (MLJ 11, 1:10,000 dilution) were incubated for one hour at room temperature. Filters were washed with TBST three times for 10 minutes each. Secondary horse radish peroxidase conjugated goat antimouse antibodies (ThermoFisher) were incubated for one hour at 1:10,000 before repeating the wash. Blots were then visualized with a chemiluminescent substrate kit (SuperSignal WestPico, Thermo Scientific).

We determined the apparent binding affinity of CadA for K. pneumoniae by measuring the amount of protein remaining unbound. K. pneumoniae was grown to an OD600=1.0 in SM. One mL of bacteria was spun down and washed with Sor buffer. The bacteria were then resuspended in 60 μl of Sor buffer with 0-70 μg of CadA and incubated for one hour. The bacteria were pelleted and 30 μl of supernatant was collected and unbound protein was measured by BioRad Protein Assay. By subtracting the unbound protein from the known input concentration, we calculated the amount of CadA bound to the bacteria. Estimated binding affinity of CadA for K. pneumoniae was calculated using the method of Steck and Wallach (Steck and Wallach, 1965).

To determine possible lectin binding activity of CadA, we repeated the above experiment in the presence 0-300 mM D-Galactose, D-Trehalose, or D-Glucosamine (EMD-Millipore). K. pneumoniae was grown as previously described. Next, bacteria, saccharide, and 10 μg CadA were mixed in 60 μl for one hour. Supernatants were collected, and unbound protein measured by the BioRad Protein Assay.

Purification of secreted antibacterials. Secreted antibacterials were purified as described before8. Briefly, 10 SM plates with AX4 growing on K. pneumoniae were scraped and amoebae separated by low-speed centrifugation. The amoebae were washed once with Sor buffer and brought up to 40 ml in Sor buffer. Overnight culture (10 ml) of K. pneumoniae grown in SM was pelleted at 4,000 RPM and added to the amoebae. The amoebae and bacteria were gently mixed on an orbital shaker for 2 hours. The sample was then spun at 1,500 RPM for 5 minutes in a TX-100 swinging bucket rotor (Sorvall Legend-RT) to pellet the amoebae. Next, the supernatant was centrifuged at 4,000 RPM for 5 minutes to pellet the bacteria. Finally, the supernatant was centrifuged at 20,000 RPM (Sorvall 4B ss34 rotor) for 10 minutes at 4° C. The supernatant was then filtered (0.22-μm pore PES filter (Corning)) and loaded onto a 10-ml DEAE sepharose CL-6B (GE Helathcare) column. The column was washed with 3 column volumes of 10 mM Tris-HCl (pH8) buffer. Elution fractions (1 ml) were then collected from 100 mM steps of NaCl in 10 mM Tris-HCl (pH8).

Antibacterial activity assay. Overnight K. pneumoniae cultures were diluted 100,000-fold in SM media and 50 μl of this dilution were put into wells of a 96-well plate. Samples (antibacterials, CadA, or antibacterials previously incubated with CadA) were then deposited in the first row of wells in a two-fold dilution to bring the total well volume to 100 μl. Each sample was then diluted across the plate two-fold. The plates were then placed at 37° C. overnight. Growth was observed as cloudy wells and checked by dilution onto SM agar plates that were placed at 37° C. overnight.

Spatial inhibition of bacterial growth. Bacteria were grown overnight at 37° C. Two μl of each bacterial suspension were spotted onto the respective bacterial nutrient agar and allowed to dry. Once dried, 10 μg of CadA or BSA was spotted adjacent but not touching the bacterial colonies and allowed to dry. After drying completely, the plates were placed in a 37° C. incubator and imaged every day for 4 days.

Agglutination. Bacteria were prepared as before in overnight cultures. One ml of OD600=1.0 bacteria was centrifuged and washed with 200 μl Sor buffer. The bacteria were then resuspended with CadA to 60 μl and incubated for 45 minutes to two hours. Bacteria were centrifuged at 7,000 rpm for 4 minutes, resuspended in 60 μl Sor buffer, and 5 μl of each bacteria were placed on slides and imaged. Removal of divalent cations was accomplished by incubating CadA with 10 mM EGTA in Sor buffer for 1 hour. EGTA was removed by buffer exchange in a 3,000 kDa molecular weight cutoff spin column (Centricon Ultracel®-3K) with Sor buffer. Mechanical agglutination was achieved by centrifugation of bacterial suspensions at 12,000 rpm for 5 minutes. Bacteria were then gently resuspended in 60 μL to maintain large aggregates.

Predation videos. Amoebae were grown to mid-log phase (1-2×106 cells/ml) in HL5 with PSV (50 U/mL penicillin and 50 μg/mL streptomycin). K. pneumoniae was grown and agglutinated as above. 5×10⁵ AX4 or cadA− amoebae were mixed with 5 μL of aggregated bacteria in 300 μl and spread onto 60 mm 1.5% KK2 (2.2 g KH₂PO₄, 0.7 g K₂HPO₄ per liter, pH 6.4) buffered agar plates. The cells were allowed to dry before 50×50 mm squares were cut and placed upside down onto glass bottomed 6 or 12-well dishes (MatTek 12-well glass bottom culture plate). To test the effect of folate on cell movement, folic acid was added to the suspension of amoeba and bacteria at 100 μM prior to deposition on the agar plate. Images were taken on Nikon Eclipse Ti using NIS Elements imaging software version 4.51.00 (build 1143). NIS Elements imaging software was used to track individual amoebae over the course of the experiment. Chemotactic index was calculated as the distance moved toward the center of a bacterial aggregate divided by the total distance (Escalante et al., 1997). Cells beginning the experiment in contact with an aggregate were excluded. Speed (FIG. 17A) and path length (FIG. 17B) were calculated by NIS Elements tracking.

Statistics and reproducibility. Non-normal distribution of plating efficiency data was determined by Shapiro-Wilk test. Statistical significance for plating efficiency was calculated by Kruskal-Wallis one-way analysis of variance followed by an ad hoc pairwise Wilcoxon rank sum test (FIG. 10B) from three biological replicates of three technical replicates. Statistical significance of exogenous CadA rescue (FIG. 10C) was calculated by Wilcoxon rank sum test. Non-normal distribution of saccharide inhibition data was determined by Shapiro-Wilk test. Statistical significance of saccharide inhibition of CadA binding (FIG. 12B) was determined by Wilcoxon rank sum test. Chemotactic index data represents n>40 cells in each condition and was non-normally distributed by Shapiro-Wilk test (FIG. 14C). Significance for chemotactic indices was determined by Wilcoxon rank sum test (FIG. 14C). Non-normal distribution of speed measurements was determined by visual inspection of quantile-quantile plots and histograms.

Statistical significance was determined by Wilcoxon rank sum test. Path Length was determined to be normally distributed by Shapiro-Wilk test. Statistical significance was determined but Welch's t-test for unequal variance (FIG. 17B).

### REFERENCES

All patents and publications mentioned in this specification are indicative of the level of those skilled in the art to which the invention pertains. All patents and publications herein are incorporated by reference to the same extent as if each individual publication was specifically and individually indicated to be incorporated by reference in their entirety.

### PUBLICATIONS

1. R. H. Kessin, Dictyostelium—Evolution, cell biology, and the development of multicellularity., (Cambridge Univ. Press, Cambridge, UK, 2001), pp. xiv+294.

2. K. B. Raper, Growth and development of Dictyostelium discoideum with different bacterial associates. J. Agr. Res. 55, 289-316 (1937).

3. G. Chen, O. Zhuchenko, A. Kuspa, Immune-like phagocyte activity in the social amoeba. Science 317, 678-681 (2007).

4. X. Zhang, O. Zhuchenko, A. Kuspa, T. Soldati, Social amoebae trap and kill bacteria by casting DNA nets. Nature communications 7, 10938 (2016).

5. D. A. Brock, T. E. Douglas, D. C. Queller, J. E. Strassmann, Primitive agriculture in a social amoeba. Nature 469, 393-396 (2011).

6. P. Stallforth et al., A bacterial symbiont is converted from an inedible producer of beneficial molecules into food by a single mutation in the gacA gene. Proc Natl Acad Sci USA 110, 14528-14533 (2013).

7. D. A. Brock, S. Read, A. Bozhchenko, D. C. Queller, J. E. Strassmann, Social amoeba farmers carry defensive symbionts to protect and privatize their crops. Nature communications 4, 2385 (2013).

8. S. DiSalvo et al., Burkholderia bacteria infectiously induce the proto-farming symbiosis of Dictyostelium amoebae and food bacteria. Proc Natl Acad Sci USA 112, E5029-5037 (2015).

9. D. A. Brock, W. E. Callison, J. E. Strassmann, D. C. Queller, Sentinel cells, symbiotic bacteria and toxin resistance in the social amoeba Dictyostelium discoideum. Proceedings. Biological sciences/The Royal Society 283, (2016).

10. S. H. Barondes, D. N. W. Cooper, W. R. Springer, in Methods in cell biology, J. A. Spudich, Ed. (Ac. Press, Orlando, Fla., 1987), vol. 28, pp. 387-409.

11. E. F. S. Wong, S. K. Brar, H. Sesaki, C. Z. Yang, C. H. Siu, Molecular cloning and characterization of DdCAD-1, a Ca2+-dependent cell-cell adhesion molecule, in Dictyostelium discoideum. J. Biol. Chem. 271, 16399-16408 (1996).

12. S. Basu, P. Fey, D. Jimenez-Morales, R. J. Dodson, R. L. Chisholm, dictyBase 2015: Expanding data and annotations in a new software environment. Genesis (New York, N.Y.: 2000) 53, 523-534 (2015).

13. L. Eichinger et al., The genome of the social amoeba Dictyostelium discoideum. Nature 435, 43-57 (2005).

14. S. V. Mathieu, K. S. Aragao, A. Imberty, A. Varrot, Discoidin I from Dictyostelium discoideum and Interactions with oligosaccharides: specificity, affinity, crystal structures, and comparison with discoidin II. J Mol Biol 400, 540-554 (2010).

15. D. N. Cooper, S. C. Lee, S. H. Barondes, Discoidin-binding polysaccharide from Dictyostelium discoideum. J. Biol. Chem. 258, 8745-8750 (1983).

16. D. N. W. Cooper, P. L. Haywood-Reid, W. R. Springer, S. H. Barondes, Bacterial glycoconjugates are natural ligands for the carbohydrate binding site of discoidin I and influence its cellular compartmentalization. Dev. Biol. 114, 416-425 (1986).

17. Simpson, Rosen, Barondes, Discoidin, a developmentally regulated carbohydrate binding protein from Dictyostelium discoideum. Purification and characterization. Biochem. 13, 3487-3493 (1974).

18. W. A. Frazier, S. D. Rosen, R. W. Reitherman, S. H. Barondes, Purification and comparison of two developmentally regulated lectins from Dictyostelium discoideum—Discoidin I and II. J. Biol. Chem. 250, 7714-7721 (1975).

19. E. Vinogradov et al., Structures of lipopolysaccharides from Klebsiella pneumoniae. Eluicidation of the structure of the linkage region between core and polysaccharide O chain and identification of the residues at the non-reducing termini of the O chains. J Biol Chem 277, 25070-25081 (2002).

20. D. W. Reitherman, S. D. Rosen, W. A. Frazier, S. H. Barondes, Cell surface species-specific high affinity receptors for discoidin: Developmental regulation in Dictyostelium discoideum. Proc. Natl. Acad. Sci. USA 72, 3541-3545 (1975).

21. S. Hirose, B. Santhanam, M. Katoh-Kurosawa, G. Shaulsky, A. Kuspa, Allorecognition, via TgrB1 and TgrC1, mediates the transition from unicellularity to multicellularity in the social amoeba Dictyostelium discoideum. Development 142, 3561-3570 (2015).

22. I. Lagarda-Diaz, A. M. Guzman-Partida, L. Vazquez-Moreno, Legume Lectins: Proteins with Diverse Applications. International journal of molecular sciences 18, 1-18 (2017).

23. M. Clarke, S. C. Kayman, K. Riley, Density-dependent induction of discoidin-I synthesis in exponentially growing cells of Dictyostelium discoideum. Differentiation 34, 79-87 (1987).

24. M. Clarke, J. Yang, S. Kayman, Analysis of the prestarvation response in growing cells of Dictyostelium discoideum. Dev. Genet. 9, 315-326 (1988).

25. I. Ofek, N. Sharon, Lectinophagocytosis: a molecular mechanism of recognition between cell surface sugars and lectins in the phagocytosis of bacteria. Infect Immun 56, 539-547 (1988).

26. A. Athamna et al., Lectinophagocytosis of encapsulated Klebsiella pneumoniae mediated by surface lectins of guinea pig alveolar macrophages and human monocyte-derived macrophages. Infect Immun 59, 1673-1682 (1991).

27. Z. Bar-Shavit, R. Goldman, Concanavalin A-mediated attachment and ingestion of yeast cells by macrophages. Exp Cell Res 99, 221-236 (1976).

28. R. Gallily, B. Vray, I. Stain, N. Sharon, Wheat germ agglutinin potentiates uptake of bacteria by murine peritoneal macrophages. Immunology 52, 679-686 (1984).

29. R. Gallily, I. Stain, O. Zaady, Dual effect of lectins on macrophages: potentiation of bacterial uptake and suppression of bactericidal activity. Immunol Lett 13, 151-158 (1986).

30. A. A. Gust, R. Willmann, Y. Desaki, H. M. Grabherr, T. Nurnberger, Plant LysM proteins: modules mediating symbiosis and immunity. Trends Plant Sci 17, 495-502 (2012).

31. X. Pang et al., Mosquito C-type lectins maintain gut microbiome homeostasis. Nat Microbiol 1, 16023 (2016).

32. M. Sussman, in Methods in Cell Physiology, D. Prescott, Ed. (Ac. Press, New York, 1966), pp. 397-409.

33. D. A. Knecht, S. M. Cohen, W. F. Loomis, H. F. Lodish, Developmental regulation of Dictyostelium discoideum actin gene fusions carried on low-copy and high-copy transformation vectors. Mol. Cell. Biol. 6, 3973-3983 (1986).

34. R. Benabentos et al., Polymorphic members of the lag gene family mediate kin discrimination in Dictyostelium. Curr Biol 19, 567-572 (2009).

35. E. A. Ostrowski, M. Katoh, G. Shaulsky, D. C. Queller, J. E. Strassmann, Kin discrimination increases with genetic distance in a social amoeba. PLoS Biol 6, e287 (2008).

36. M. Sussman, Cultivation and synchronous morphogenesis of Dictyostelium under controlled experimental conditions. Meth. Cell Biol. 28, 9-29 (1987).

37. M. L. Knetsch, G. Tsiavaliaris, S. Zimmermann, U. Ruhl, D. J. Manstein, Expression vectors for studying cytoskeletal proteins in Dictyostelium discoideum. Journal of muscle research and cell motility 23, 605-611 (2002).

38. H. Towbin, T. Staehelin, J. Gordon, Electrophoretic transfer of proteins from polyacrylamide gels to nitrocellulose sheets: procedure and some applications. Proc Natl Acad Sci USA 76, 4350-4354 (1979).

39. B. Wetterauer, G. Jacobsen, P. Morandini, H. MacWilliams, Mutants of Dictyostelium discoideum with defects in the regulation of discoidin-I expression. Dev. Biol. 159, 184-195 (1993).

40. G. Gerisch, O. Luderitz, E. Ruschmann, Antikorper fordern die Phagozytose von Bacterien durch Amoben. Z Naturforsch. 22B, 109 (1967).

41. M. M. Bradford, A rapid and sensitive method for the quantitation of microgram quantities of protein utilizing the principle of protein-dye binding. Anal Biochem 72, 248-254 (1976).

42. J. Fox, The R Commander: A Basic Statistics Graphical User Interface to R. Journal of Statistical Software 14, 1-42 (2005).

43. S. D. Rosen, J. A. Kafka, D. L. Simpson, S. H. Barondes, Developmentally regulated, carbohydrate-binding protein in Dictyostelium discoideum. Proc. Natl. Acad. Sci. USA 70, 2554-2557 (1973).

44. T. Lyle Steck, D. F. Hoelzl Wallach, The binding of kidney-bean phytohemagglutinin by Ehrlich ascites carcinoma. Biochimica et Biophysica Acta (BBA)—General Subjects 97, 510-522 (1965).

45. H. P. Erickson, Size and shape of protein molecules at the nanometer level determined by sedimentation, gel filtration, and electron microscopy. Biol Proced Online 11, 32-51 (2009).

46. G. Chen, G. Shaulsky, A. Kuspa, Tissue-specific G1-phase cell-cycle arrest prior to terminal differentiation in Dictyostelium. Development 131, 2619-2630 (2004).

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. A method of improving a microbiome in a subject, comprising the step of providing to the subject an effective amount of a composition comprising a plurality of one or more types of isolated bacteria having one or more types of lectins externally associated thereto, said bacteria formulated in a pharmaceutically acceptable carrier.

2. The method of claim 1, wherein the subject is an animal or plant.

3. The method of claim 2, wherein the animal is a livestock animal or farm animal.

4. The method of claim 2, wherein the animal is a mammal.

5. The method of claim 4, wherein the mammal is a human, dog, cat, horse, lamb, sheep, pig, goat, rabbit, or cow.

6. The method of claim 4, wherein the mammal is a human and the human is an adult, adolescent, child, or infant.

7. The method of claim 2, wherein the animal is a fish or bird.

8. The method of claim 2, wherein the plant is an agricultural plant or ornamental plant.

9. The method of claim 1, wherein the bacteria are Gram negative, Gram positive, aerobic, anaerobic, or a mixture thereof.

10. The method of claim 1, wherein the subject is healthy.

11. The method of claim 1, wherein the subject has a medical condition.

12. The method of claim 11, wherein the medical condition is a gastrointestinal medical condition or an autoimmune medical condition.

13. The method of claim 1, wherein the bacteria are nonpathogenic bacteria selected from the group consisting of Bifidobacterium, Clostridium, Fusobacterium, Melissococcus, Propionibacterium, Streptococcus, Enterococcus, Lactococcus, Staphylococcus, Peptostrepococcus, Bacillus, Pediococcus, Micrococcus, Leuconostoc, Weis sella, Aerococcus, Oenococcus, Lactobacillus, Bacteriodetes, Prevotella, Bacteroides, Firmicutes, Xylanibacter, Faecalibacterium, Eubacterium, Subdoligranulum, Parabacteriodetes, Ruminococcus, Collinsella, Rosehburia, Akkermansia, Veillonella, Verrucomicrobia, Escherichia, and a mixture thereof.

14. The method of claim 13, wherein the bacteria comprise Bifidobacterium and/or Lactobacillus.

15. The method of claim 1, wherein the bacteria are selected from the group consisting of Bifidobacterium animalis, Escherichia coli, Lactococcus lactis, Lactobacillus reuteri, Tobacillus acidophilus, Acidophilus bifidus, Streptococcus thermophiles, Streptococcus faecium, Clostridium leptum, Clostridium coccoides, Bacillus coagulans, B. lentus, B. licheniformis, B. mesentericus, B. pumilus, B. subtilis, B. natto, Bacteroides amylophilus, Bac. capillosus, Bac. ruminocola, Bac. suis, Bifidobacterium adolescentis, B. animalis, B. breve, B. bifidum, B. infantis, B. lactis, B. longum, B. pseudolongum, B. thermophilum, Candida pintolepesii, Clostridium butyricum, Enterococcus cremoris, E. diacetylactis, E. faecium, E. intermedius, E. lactis, E. muntdii, E. thermophilus, Escherichia coli, Kluyveromyces fragilis, Lactobacillus acidophilus, L. alimentarius, L. amylovorus, L. crispatus, L. brevis, L. case L. curvatus, L. cellobiosus, L. delbrueckii ss. bulgaricus, L farciminis, L. fermentum, L. gasseri, L. helveticus, L. lactis, L. plantarum, L. johnsonii, L. reuteri, L. rhamnosus, L. sakei, L. salivarius, Leuconostoc mesenteroides, P. cereviseae (damnosus), Pediococcus acidilactici, P. pentosaceus, Propionibacterium freudenreichii, Prop. shermanii, Saccharomyces cereviseae, Staphylococcus carnosus, Staph. xylosus, Streptococcus infantarius, Strep. Salivarius, Strep. thermophilus, Strep. lactis, and a mixture thereof.

16. The method of claim 1, wherein the lectin is a mannose binding lectin, galactose/N-acetylgalactosamine binding lectin, N-acetylglucosamine binding lectin, N-acetylneuraminic acid binding lectin, or Fucose binding lectin.

17. The method of claim 1, wherein the lectin is a C type lectin, L type lectin R type lectin, or P type lectin.

18. The method of claim 1, wherein the lectin is Galectin, Concanavalin A, collectin, selectin Lentil lectin, Snowdrop lectin, Ricin, Peanut agglutinin, Jacalin, Hairy vetch lectin, Wheat Germ Agglutinin, Elderberry lectin, Maackia amurensis leukoagglutinin, Maackia amurensis hemoagglutinin, Ulex europaeus agglutinin, Aleuria aurantia lectin, BanLec, Achylectin, Anti-B agglutinin, calnexin, calreticulin, or a mixture thereof.

19. The method of claim 1, wherein the subject is an animal and said method further comprises the step of the subject consuming one or more prebiotics.

20. The method of claim 1, further comprising the step of identifying that the subject is in need of the composition.

21. The method of claim 20, wherein the individual has a medical condition in need of treatment.

22. The method of claim 21, wherein the medical condition is a gastrointestinal medical condition or an autoimmune medical condition.

23. A method of protecting one or more types of bacteria from destruction by one or more antibacterial agents or from destruction by an environment, comprising the step of exposing the one or more types of bacteria to an effective amount of one or more types of lectins to produce bacteria having the one or more types of lectins externally associated thereto, whereby upon exposure to the one or more antibacterial agents and/or the environment, said one or more types of bacteria are protected.

24. The method of claim 23, wherein the antibacterial agent is a protein.

25. The method of claim 23, wherein the antibacterial agent is a protein from one or more types of pathogenic bacteria.

26. The method of claim 23, wherein the environment is a gastrointestinal system of an animal.

27. A method of producing a probiotic composition, comprising the steps of:
exposing one or more types of bacteria to one or more types of lectin under conditions sufficient to result in binding of the lectin(s) to the bacteria to produce lectin-bound bacteria; and
formulating the lectin-bound bacteria in a pharmaceutically acceptable carrier.

28. A method of facilitating uptake of one or more types of bacteria into one or more types of cells, comprising the step of binding one or more types of lectins to the bacteria in a sufficient amount for the bacteria to be uptaken into the cell.

29. The method of claim 28, wherein the lectin is derived from plant, amoeba, animal, or a mixture thereof.

30. The method of claim 28, wherein the bacteria is Gram-negative or Gram-positive.

31. The method of claim 23, wherein the bacteria is pathogenic or non-pathogenic.

32. The method of claim 28, wherein the bacteria is spherical, rod shaped, or spiral.

33. The method of claim 28, wherein the bacteria is of the type Coccus, Bacillus, Vibrio, Spirillum, or Spirochete.

34. The method of claim 28, wherein the bacteria is genetically engineered.

35. The method of claim 28, wherein the bacteria comprises a genetically engineered mutation.

36. The method of claim 28, wherein the cell is a mammalian cell

37. The method of claim 36, wherein the mammalian cell is a cancer cell.

38. The method of claim 36, wherein the mammalian cell is a human cell.

39. The method of claim 36, wherein the mammalian cell is an immune cell or an epithelial cell.

40. The method of claim 36, wherein the mammalian cell is a primary cell, an established cell line, or a hybridoma cell.

41. The method of claim 36, wherein the mammalian cell is a germ cell, somatic cell, stem cell, progenitor cell, pluripotent cell, totipotent cell, or a mixture thereof.

42. The method of claim 36, wherein the mammalian cell is from the ectoderm, mesoderm, or endoderm.

43. The method of claim 28, wherein the lectin is mannose binding, Galactose/N-acetylgalactosamine binding, N-acetylglucosamine binding, N-acetylneuraminic acid binding, or fucose binding.

44. The method of claim 28, wherein the bacteria is E. coli.

45. The method of claim 28, wherein the method occurs ex vivo, in vivo, or in vitro.

46. The method of claim 28, wherein the cell is in culture.
